(19) United States
(12) Patent Application Publication
(10) Pub. No.: US 2026/0240782 A1
(43) Pub. Date: Aug. 20, 2026

(54) PALATABLE CHEWABLE COMPOSITIONS WITH IMPROVED TASTE MASKING

(71) Applicant: Harmony Foods LLC, Santa Cruz, CA (US)

(72) Inventors: April Anthony, Concord, NC (US); Rachel Leah Hadley, Davidson, NC (US); Andrew Wassel, Mooresville, NC (US); Scott David Vogt, Aptos, CA (US); Jacob Lee Orlowicz, San Jose, CA (US); Michael Alan Simpson, Mooresville, NC (US); Perry Nga, Dublin, CA (US)

(21) Appl. No.: 19/647,159

(22) Filed: Apr. 14, 2026

Related U.S. Application Data
(62) Division of application No. 19/443,532, filed on Jan. 8, 2026.
(60) Provisional application No. 63/743,185, filed on Jan. 8, 2025.

(51) Int. Cl. A61K 9/48; A61K 9/00; A61K 31/167; A61K 31/20; A61K 31/522; A61K 31/616

(52) U.S. Cl. CPC A61K 9/48; A61K 9/0056; A61K 31/167; A61K 31/20; A61K 31/522; A61K 31/616

(57) ABSTRACT

The present disclosure is directed to improved palatable chewable composition products that contain at least one encapsulated primary ingredient. Encapsulation prevents unpalatable flavors or unpleasant odors from the primary ingredients during ingestion. The disclosed palatable chewable compositions maintain palatability through manufacturing and shelf life.

### CROSS-REFERENCE TO RELATED APPLICATIONS

This application is a divisional of U.S. application Ser. No. 19/443,532, filed Jan. 8, 2026, which claims priority to U.S. Provisional Application No. 63/743,185 filed Jan. 8, 2025, the entire contents of which are incorporated by reference.

### FIELD OF THE DISCLOSURE

The present disclosure relates to a palatable chewable composition that contains one or more primary ingredients and demonstrates taste masking. The present disclosure also relates to methods, manufacturing systems, and methods of use related to the palatable chewable composition.

### BACKGROUND

Palatable chewable compositions have become popular tools for providing dietary supplements and various medications in an easy-to-ingest vehicle. However, for effective delivery of many primary ingredients, there are significant unmet needs to be overcome. By way of example only, some primary ingredients may have unpleasant flavors, aromas, irritations, or sensations that might discourage ingestion. This may include alkaloids such as caffeine or other xanthines, numerous bitter medicinal compounds, the flavor of many whey compounds and other dietary supplements such as vitamins, minerals such as iron or its salts or complexes, plant materials such as garlic extracts, phenols and polyphenols, flavonoids (e.g., epicatechin gallate from green tea or other catechins, naringin from citrus), terpenes, isoflavones, and animal-derived compounds such as fish oil or fish extract. There is a need for improved methods of managing unpleasant flavors of primary ingredients or unpleasant odors without interfering with the intended delivery of the primary ingredients, while also being compatible with the challenging environment experienced during manufacturing and then during storage. One or more of the methods, products, systems, compositions, and means of manufacture described herein may in some way address one or more of the exemplary needs noted here, but they need not solve the associated problems or meet any specific need listed here in order to be within the scope of the claims or to provide valuable benefits.

### BRIEF SUMMARY

The present disclosure relates to palatable chewable compositions for masking the taste of a primary ingredient. In some aspects, the palatable chewable composition may comprise an encapsulated primary ingredient selected from an active pharmaceutical ingredient, a dietary supplement, a food, a food additive, or combinations thereof. The encapsulated primary ingredient may be present in the palatable chewable composition in an amount from about 0.05% to about 35% by weight of the composition.

In some aspects, the composition may further comprise a matrix in which the encapsulated primary ingredient may be dispersed. The matrix may be present in an amount from about 60% to about 96% by weight of the composition. In some aspects, the matrix may include a sweetener and a solvent. In other aspects, the matrix may include a sweetener or a bulking agent, a solvent, and optionally a hydrocolloid. When present, the hydrocolloid may be included in an amount from about 0.5% to about 15% by weight of the composition. The solvent, when present, may be included in an amount from about 10% to about 30% by weight of the composition.

The present disclosure relates to a palatable chewable composition for masking the taste of a primary ingredient. The palatable chewable composition may include an encapsulated primary ingredient selected from the group consisting of an active pharmaceutical ingredient, a dietary supplement, a food, and any combination thereof. The encapsulated primary ingredient may be present in the palatable chewable composition in an amount from about 0.05% to about 35% by weight of the composition. The palatable chewable composition may also include a matrix in an amount from about 60% to about 96% by weight of the composition. The matrix may include a sweetener, a hydrocolloid in an amount from about 0.5% to about 15% by weight of the composition, and a solvent in an amount from about 10% to about 30% by weight. In some aspects, the encapsulated primary ingredient may be dispersed in the matrix.

The palatable chewable composition may include an encapsulated primary ingredient selected from the group consisting of an active pharmaceutical ingredient, a dietary supplement, a food, and any combination thereof. The palatable chewable composition may also include a matrix. The matrix may include a sweetener, a hydrocolloid, and a solvent. In some aspects, the encapsulated primary ingredient may be dispersed in the matrix. The encapsulated primary ingredient may be dispersed in the matrix in an amount from about 0.05% to about 35% by weight of the palatable chewable composition.

The palatable chewable composition may include an encapsulated primary ingredient selected from the group consisting of an active pharmaceutical ingredient, a dietary supplement, a food, and any combination thereof. The encapsulated primary ingredient may be present in the palatable chewable composition in an amount from about 0.05% to about 35% by weight of the composition. The palatable chewable composition may also include a matrix in an amount from about 60% to about 96% by weight of the composition. The matrix may include a sweetener and a solvent in an amount from about 10% to about 30% by weight. In some aspects, the encapsulated primary ingredient may be dispersed in the matrix.

The disclosed compositions provide effective taste masking of the encapsulated primary ingredient while maintaining a palatable chewable dosage form with desirable texture and consumer acceptability.

The present disclosure further relates to methods of making palatable chewable compositions for masking the taste of a primary ingredient. In some aspects, the method may comprise combining a sweetener, a hydrocolloid, and a solvent to form a homogeneous mixture. An encapsulated primary ingredient may then be added to the homogeneous mixture while mixing, wherein the primary ingredient may be selected from an active pharmaceutical ingredient, a dietary supplement, a food additive, or combinations thereof. The homogeneous mixture may subsequently be heated or conditioned to a temperature from about 40° C. to about 70° C. and deposited into molds. The deposited mixture may then be cooled to form the chewable composition. The disclosed method provides a chewable dosage form with improved taste masking, uniform dispersion of the encapsulated primary ingredient, and desirable texture and palatability.

In some aspects, the method may comprise combining a sweetener and a solvent to form a homogeneous mixture. An encapsulated primary ingredient may then be added to the homogeneous mixture while mixing, wherein the primary ingredient may be selected from an active pharmaceutical ingredient, a dietary supplement, a food additive, or combinations thereof. The homogeneous mixture may subsequently be conditioned to a temperature from about 40° C. to about 70° C. and deposited into molds. The deposited mixture may then be cooled to form the chewable composition. The disclosed method enables effective taste masking of the primary ingredient while providing a chewable dosage form with desirable texture and palatability.

In some aspects, the method may comprise combining a sweetener, a hydrocolloid, and a solvent to form a homogeneous mixture. An encapsulated primary ingredient may be added to the homogeneous mixture while mixing, wherein the primary ingredient may be selected from an active pharmaceutical ingredient, a dietary supplement, a food additive, or combinations thereof. The homogeneous mixture may be heated to a temperature of up to about 95° C. and deposited into molds within about 40 minutes from the time the homogeneous mixture is heated. The deposited mixture may then be cooled to form the chewable composition. The disclosed method provides effective taste masking of the primary ingredient while producing a chewable dosage form with desirable texture and palatability.

In some aspects, the method may comprise combining a sweetener, a hydrocolloid, and a solvent to form a homogeneous mixture. An encapsulated primary ingredient may then be added to the homogeneous mixture while mixing, wherein the primary ingredient may be selected from an active pharmaceutical ingredient, a dietary supplement, a food additive, or combinations thereof. The homogeneous mixture may be heated to a temperature that does not exceed a decomposition temperature of the primary ingredient and may be subsequently deposited into molds. The deposited mixture may then be cooled to form the chewable composition. The disclosed method facilitates effective taste masking while preserving the stability of the primary ingredient and providing a chewable dosage form with desirable texture and palatability.

The present disclosure further relates to methods of making palatable chewable compositions for masking the taste of a primary ingredient. In some aspects, the method may include combining one or more sweeteners with a solvent to form a homogeneous mixture. In some aspects, the method may further include combining a hydrocolloid with the sweetener and the solvent. An encapsulated primary ingredient may be added to the homogeneous mixture while mixing, wherein the primary ingredient may be selected from an active pharmaceutical ingredient, a dietary supplement, a food additive, or combinations thereof.

In some aspects, the homogeneous mixture may be conditioned or heated to an elevated temperature to facilitate processing, uniformity, and moldability. The temperature may range from about 40° C. to about 70° C., may be up to about 95° C., or may be selected such that it does not exceed a decomposition temperature of the encapsulated primary ingredient. In some aspects, the homogeneous mixture may be deposited into molds within a predetermined time period following heating, such as within about 40 minutes.

The deposited mixture may then be cooled to form the chewable composition. The disclosed methods enable effective taste masking of the primary ingredient while maintaining the stability of the encapsulated primary ingredient and producing a chewable dosage form with desirable texture, uniformity, and palatability.

The present disclosure further relates to methods of making palatable chewable compositions for masking the taste of a primary ingredient. In some aspects, the method may include combining a sweetener and a solvent to form a homogeneous mixture. In some aspects, the next step of the method may include adding an encapsulated primary ingredient to the homogeneous mixture while mixing. In some aspects, the method may include conditioning the homogeneous mixture to a temperature from about 40° C. to about 95° C. The last step of the method may include forming the homogeneous mixture into shapes to form the chewable composition.

### BRIEF DESCRIPTION OF THE FIGURES

Preferred embodiments of the invention are described in detail below with reference to the attached figures, wherein:

FIG. 1A depicts a multicomponent palatable chewable composition with enhanced stability for one or more primary ingredients in accordance with one embodiment.

FIG. 1B depicts a multicomponent palatable chewable composition with enhanced stability for one or more primary ingredients in accordance with one embodiment.

FIG. 2A depicts cross-sections of palatable chewable composition form products in accordance with one embodiment.

FIG. 2B depicts cross-sections of palatable chewable composition form products in accordance with one embodiment.

FIG. 3 is a bar graph depicting the bitterness intensity of acetaminophen soft chewable tablets according to Example 1.

FIG. 4 is a line graph depicting the bitterness intensity and the sweetness profile of acetaminophen soft chewable tablets according to Example 1.

FIG. 5A is a radar plot depicting initial sensory scores for fruity flavor, sweetness, bitterness, and medicinal of the disclosed palatable chewable composition compared to commercially available products in accordance with one embodiment.

FIG. 5B is a radar plot depicting aftertaste sensory scores for fruity flavor, sweetness, bitterness, and medicinal of the disclosed palatable chewable composition compared to commercially available products in accordance with one embodiment.

FIG. 6 is a plot illustrating the percentage of APAP released as a function of time during high-shear processing (Turrax at 2800 RPM) in accordance with one embodiment.

FIG. 7 is a contour plot showing free acetaminophen (APAP) concentration (%) as a function of processing temperature (° C.) and exposure time(s) under fixed shear rate (600 rpm) in a water-based system in accordance with one embodiment.

FIG. 8 is a plot showing percent free APAP release over a 40-minute thermal hold for five disclosed systems in Example 4.

FIG. 9A is a photograph of the control pectin formulation (Run 5, 85° C. with acid) after a 40-minute thermal hold, showing visual evidence of vertical stratification.

FIG. 9B is a plot showing APAP assay results from samples collected at the top and bottom of the vessel over a 40-minute hold.

FIG. 10 is a plot showing time-dependent caffeine concentrations for three replicate runs of the coated system, demonstrating consistent release behavior and low variability relative to the nominal concentration limit in accordance with one embodiment.

FIG. 11 is a bar graph showing comparative sensory intensities for free vs. encapsulated ibuprofen soft chews, showing reduced bitterness, astringency, and mouth burn in the encapsulated formulation in accordance with one embodiment.

FIG. 12A is a bar plot showing descriptive analysis (DA) panel scores for initial chew intensity across four sensory attributes in accordance with one embodiment.

FIG. 12B is a bar plot showing descriptive analysis (DA) panel scores for aftertaste intensity ratings collected 30 seconds post-expectoration for fruity flavor, medicinal notes, sweetness, and bitterness across three sample types: control soft chew, encapsulated APAP soft chew, and free (non-encapsulated) APAP in accordance with one embodiment.

### DETAILED DESCRIPTION

The present disclosure relates to a palatable chewable composition for masking the taste of a primary ingredient. The palatable chewable composition may be in the form of a gummy, chewable gel, or soft chew matrix. The palatable chewable composition may include an encapsulated primary ingredient. The encapsulated primary ingredient may be dispersed in a matrix. The primary ingredient may include an active pharmaceutical ingredient, a dietary supplement, a food, or any combination thereof. The matrix may include a sweetener, a hydrocolloid, and a solvent. The matrix may further include a humectant, a color additive, a filler, a lipid, an emulsifier, a flavor enhancer, a flavor masking agent, a pH modifier, a flavor modifier, or any combination thereof. The palatable chewable composition may be a chewable gel.

The present disclosure further relates to a palatable chewable composition that includes an active pharmaceutical ingredient. The palatable chewable composition may improve patient compliance, particularly among pediatric and taste-sensitive populations.

The present disclosure further relates to a palatable chewable composition, methods of making the palatable chewable composition, and methods of administering the palatable chewable composition. The palatable chewable composition has an improved taste-masking capability as compared to other chewable compositions known in the art. The taste-masking capability may be achieved via encapsulation of the primary ingredient dispersed in the matrix of the palatable chewable composition. The encapsulation may further enable moisture protection of the primary ingredient, while still allowing for dissolution of the primary ingredient, particularly when a release profile of the primary ingredient is desired.

### Palatable Chewable Composition

The palatable chewable composition includes an encapsulated primary ingredient. The encapsulated primary ingredient may include an encapsulant (also referred to herein as a coating or coating layer) and a primary ingredient. The weight ratio of the encapsulant to the primary ingredient may be from about 3:97 to about 90:10. The weight ratio may be from about 3:97 to about 80:20, from about 3:97 to about 70:30, from about 3:97 to about 60:40, from about 3:97 to about 50:50, from about 3:97 to about 40:60, from about 3:97 to about 30:70, from about 3:97 to about 20:80, or from about 3:97 to about 10:90. In some embodiments, the weight ratio may be from about 3:97 to about 90:10, about 10:90 to about 90:10, about 20:80 to about 90:10, about 30:70 to about 90:10, about 40:60 to about 90:10, about 50:50 to about 90:10, about 60:40 to about 90:10, about 70:30 to about 90:10, or about 80:20 to about 90:10. The weight ratio may be from about 3:97 to about 10:90, about 3:97 to about 20:80, about 3:97 to about 30:70, about 3:97 to about 40:60, about 3:97 to about 50:50, about 3:97 to about 60:40, about 3:97 to about 70:30, about 3:97 to about 80:20, about 3:97 to about 90:10, about 20:80 to about 30:70, 20:80 to about 40:60, 20:80 to about 50:50, 20:80 to about 60:40, 20:80 to about 70:30, about 20:80 to about 80:20, about 20:80 to about 90:10, about 30:70 to about 40:60, about 30:70 to about 50:50, about 30:70 to about 60:40, about 30:70 to about 70:30, about 30:70 to about 80:20, about 30:70 to about 90:10, about 40:60 to about 50:50, about 40:60 to about 60:40, about 40:60 to about 70:30, about 40:60 to about 80:20, about 40:60 to about 90:10, about 50:50 to about 60:40, about 50:50 to about 70:30, about 50:50 to about 80:20, about 50:50 to about 90:10, about 60:40 to about 70:30, about 60:40 to about 80:20, 60:40 to about 90:10, about 70:30 to about 80:20, about 70:30 to about 90:10, or about 80:20 to about 90:10.

Primary ingredients may include active pharmaceutical ingredients (APIs). Examples of APIs include but are not limited to NSAIDs, antibiotics, anticoagulants and antiplatelet agents, antidiabetic agents, antiemetics, antihistamines, anti-asthmatic medications, antiarrhythmics, anticholinergics, anticholinesterase inhibitors, anticonvulsants, antidepressants, antidiabetic agents, antihistamines, antihypertensives (ACE inhibitors, alpha blockers, angiotensin II receptor blockers, beta blockers, calcium channel blockers, central alpha agonists, diuretics, and the like), anti-inflammatory drugs, antipsychotics, antipyretics, appetite suppressants, bronchodilators, chemotherapy compounds, cholesterol level medications (bile acid sequestrants, cholesterol absorption inhibitor such as ezetimibe, fibrates, PCSK9 inhibitors, statins, angiotensin II receptor blockers, beta blockers, calcium channel blockers, central alpha agonists, and the like), diuretics, expectorants, hormonal medications, laxatives, sedatives, stimulants, vasodilators, anxiolytics, mood stabilizers, antipsychotics, antidepressants and antipsychotics, and the like.

Although certain ingredients, such as acetaminophen, diphenhydramine, azithromycin, ibuprofen, guaifenesin, dimenhydrinate, amphetamine, fexofenadine, aspirin, dextromethorphan, amoxicillin, cetirizine, and/or methylphenidate, may appear in the examples provided below, other types of primary ingredients may be used. In some embodiments, the primary ingredient may be acetaminophen.

Aspects of the present disclosure may be equally applicable to gummy compositions where the primary ingredient is selected from the group comprising of anti-inflammatory actives, coronary dilators, cerebral dilators, peripheral vasodilators, anti-infectives, psychotropics, antimanics, stimulants, gastro-intestinal sedatives, antidiarrheal primary ingredients, anti-anginal drugs, vasodilators, anti-hypertensive drugs, vasoconstrictors and anti-migraine treatment actives, antibiotics, tranquilizers, antipsychotics, antitumor drugs, anticoagulants and antithrombotic drugs, hypnotics, sedatives, anti-emetics, antinauseants, anticonvulsants, neuromuscular drugs, hyper- and hypoglycemic agents, thyroid and antithyroid preparations, diuretics, antispasmodics, uterine relaxants, nutritional additives, anti-obesity drugs, anabolic drugs, erythropoietic drugs, antiasthmatics, expectorants, cough suppressants, mucolytics, anti-hyperuricemia drugs, antidepressants, and the like, and combinations of the foregoing.

Primary ingredients may further include nutraceuticals or other dietary supplements and natural or synthetic aids for health and beauty. Primary ingredients may include nutrient supplementation including but not limited to vitamins and/or minerals for the entire spectrum of vitamins (e.g. A, B₁, B₂, B₃, B₅, B₆, B₁₂, C, D, E, K, niacin, folate or folic acid, and the like) and minerals such as iron, calcium, copper, magnesium, selenium, iodine, and zinc, as well as any other nutraceuticals and probiotics (e.g., Lactobacillus and Bifidobacterium). Further examples include but are not limited to omega-3 fatty acids, choline, antacids such as calcium carbonate or magnesium hydroxide, fiber supplements, herbal extracts such as echinacea for immune support or chamomile for calmness and sleep, or St. Johns Wort for mental wellbeing, essential amino acids or branched-chain amino acids that may support muscle health and recovery, digestive enzymes such as papain, bromelain, lactase, peptinase, and serrapeptase that may aid in food digestion, and/or other enzymes such as nattokinase may be used for a wide variety of health purposes; laxatives; sleep aids such as melatonin; antioxidants such as N-acetyl cysteine or glutathione.

Primary ingredients may further include food ingredients that benefit from encapsulation to improve palatability, suppress undesirable taste attributes, reduce harsh chemical sensations, or otherwise enhance consumer acceptability. Food ingredients may include bitter compounds. Examples of bitter compounds include but are not limited to caffeine, green tea extract, guarana, cocoa polyphenols, ginseng extract, ashwagandha, and other botanical extracts known to produce bitterness or astringency. Examples of other food ingredients include vegetable concentrates and powders, bitter melon, cruciferous vegetables, mustard greens, fish, rhizomes such as ginger/galangal/turmeric, beetroot, cranberry, fermented foods such as kimchi, or berberine.

Additional encapsulatable food components may include strong-odor ingredients. Examples of strong-odor ingredients include garlic, onion, turmeric, fenugreek, ginger, fish oil, algal oil, or other volatile oils that benefit from odor containment and controlled release.

Encapsulation may improve the organoleptic performance of acids and souring agents by preventing harsh upfront sourness or “acid burn.” In some embodiments, the food may include citric acid, malic acid, lactic acid, and vinegar powders.

In some embodiments, the primary ingredients may include minerals. The encapsulation may reduce metallic, salty, or chalky off-notes. Examples of minerals include but are not limited to iron, zinc, magnesium, calcium salts, or potassium chloride.

High-intensity sweeteners, including Stevia and rebaudioside extracts, saccharin, acesulfame potassium, aspartame, and sucralose, may be encapsulated to suppress late-stage bitterness or chemical aftertaste.

Each of these compounds may be used in combination with other primary ingredients, with suitable coatings for each primary ingredient in order to be protected during manufacture and storage, and, in some embodiments, in order to reach their respective physiological targets by being released in the appropriate portions of the GI tract.

In referring to specific nutraceutical or pharmaceutical compounds, salts, chelates, and other related forms of the compound may be used, including derivatives thereof if the derivative is efficacious in a similar manner. Thus, for a palatable chewable composition said to contain vitamin C, that vitamin may be understood to include ascorbic acid and various forms of ascorbic acid such as ascorbates (calcium or sodium ascorbate), ascorbyl palmitate, ascorbyl phosphates and salts (such as sodium, potassium, or magnesium ascorbyl phosphate), dehydroascorbic acid and salts, ascorbyl palmitate compounds, tetrahexyldecyl ascorbate, ascorbyl sulfates and salts, acylated ascorbic acid derivatives (e.g., 6-O-acyl-2-O-alpha-D-glucopyranosyl-L-ascorbic acids), 6-bromo-6-deoxy-L-ascorbic acid, and the like.

The amount of primary ingredient in the encapsulated primary ingredient may correspond to the mass of individual finely ground particles that may be effectively ground, as well as agglomerations of such particles or of such coated particles. The total mass of a primary ingredient in a palatable chewable composition may also correspond to a reasonable dose when the primary ingredient includes an active pharmaceutical ingredient or a dietary supplement, recognizing that more than one palatable chewable composition may need to be administered to deliver a suitable dose of the primary ingredient during one instance of administering the palatable chewable composition.

In some aspects, the amount of the encapsulated primary ingredient in a palatable chewable composition may be from about 0.02 mg to about 1550 mg, about 0.5 mg to about 1500 mg, about 1 mg to about 1450 mg, about 5 mg to about 1400 mg, about 10 mg to about 1350 mg, about 15 mg to about 1300 mg, about 20 mg to about 1250 mg, about 25 mg to about 1200 mg, about 30 mg to about 1150 mg, about 35 mg to about 1100 mg, about 40 mg to about 1150 mg, about 45 mg to about 1000 mg, about 50 mg to about 900 mg, about 100 mg to about 800 mg, about 200 mg to about 700 mg, about 300 mg to about 600 mg, or about 400 mg to about 500 mg. In other aspects, recommended values for daily intake may be used such as the Recommended Dietary Allowance (RDA), Dietary Reference Intake (DRI), Reference Daily Intakes (RDIs), and Daily Values (% DV) may be considered from about 10% to about 600%, about 20% to about 400%, about 30% to about 200%, or about 50% to about 150% of the recommended values (particularly the values in force as of Jul. 1, 2023) may be given. In the example ranges here, any lower limit may be combined with any listed upper limit as if explicit support for that limit had been provided, as long as the resulting combinations are logical or feasible with the upper limit higher than the lower limit.

In some aspects, the primary ingredients include but are not limited to odorous materials such as N-acetyl cysteine, DHA, EPA (eicosapentaenoic acid), garlic, durian, cruciferous vegetables or extracts thereof, fish oil or fish extract.

In some aspects, the primary ingredient may include bitter or unpalatable agents such as alkaloids including caffeine or other xanthines, whey and collagen compounds, dietary supplements such as iron or its salts or complexes or various vitamins such as the B vitamins, plant materials such as garlic extracts, phenols and polyphenols, flavonoids (e.g., epicatechin gallate from green tea or other catechins, naringin from citrus), terpenes, isoflavones, and animal-derived compounds such as fish oil or fish extract.

Primary ingredients or matrix ingredients may also include dietary fiber such as water-soluble dietary fiber, polydextrose, inulin, indigestible dextrin, and/or various polysaccharides.

The encapsulated primary ingredient may comprise analgesics or antipyretics. In some embodiments, the analgesics may include non-steroidal anti-inflammatory drugs (NSAIDs). In some embodiments, NSAIDs may comprise acetylsalicylic acid, ibuprofen, naproxen, ketoprofen, flurbiprofen, fenoprofen, diclofenac, indomethacin, ketorolac, etodolac, sulindac, or tolmetin.

The encapsulated primary ingredient may be present in the palatable chewable composition in an amount from about 0.05% to about 40% by weight of the palatable chewable composition. For example, the encapsulated primary ingredient may be from about 0.05 to about 1%, about 0.05% to about 2%, about 0.05% to about 5%, about 0.05% to about 10%, about 0.05% to about 15%, about 0.05% to about 20%, about 0.05% to about 25%, about 0.05% to about 30%, and about 0.05% to about 35%, about 0.1% to about 1%, about 0.1% to about 2%, about 0.1% to about 5%, about 0.1% to about 10%, about 0.1% to about 15%, about 0.1% to about 20%, about 0.1% to about 25%, about 0.1% to about 30%, about 0.1% to about 35%, about 0.5% to about 2%, about 0.5% to about 5%, about 0.5% to about 10%, about 0.5% to about 15%, about 0.5% to about 20%, about 0.5% to about 25%, about 0.5% to about 30%, about 0.5% to about 35%, about 1% to about 5%, about 1% to about 10%, about 1% to about 15%, about 1% to about 20%, about 1% to about 25%, about 1% to about 30%, about 1% to about 35%, about 2% to about 5%, about 2% to about 10%, about 2% to about 15%, about 2% to about 20%, about 2% to about 25%, about 2% to about 30%, about 2% to about 35%, about 5% to about 10%, about 5% to about 15%, about 5% to about 20%, about 5% to about 25%, about 5% to about 30%, about 5% to about 35%, about 10% to about 15%, about 10% to about 20%, about 10% to about 25%, about 10% to about 30%, about 10% to about 35%, about 15% to about 20%, about 15% to about 25%, about 15% to about 30%, about 15% to about 35%, about 20% to about 25%, about 20% to about 30%, about 20% to about 35%, about 25% to about 30%, about 25% to about 35%, or from about 30% to about 35% by weight of the palatable chewable composition. As another example, the weight percent of the encapsulated primary ingredient in the palatable chewable composition may be from about 5% to about 25%, about 6% to about 24%, about 7% to about 23%, about 8% to about 22%, about 9% to about 21%, about 10% to about 20%, about 11% to about 19%, about 12% to about 18%, about 13% to about 17%, or about 14% to about 16% by weight of the palatable chewable composition. As yet another example, the encapsulated primary ingredient may be present in the palatable chewable composition in an amount of about 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or about 40% by weight of the palatable chewable composition.

In some embodiments, the encapsulated primary ingredient may be present in the palatable chewable composition from about 0.05% to about 0.1%, about 0.05% to about 0.5%, about 0.05% to about 1%, about 0.05% to about 2%, about 0.05% to about 5%, about 0.05% to about 7%, about 0.05% to about 10%, about 0.05% to about 12%, about 0.05% to about 15%, about 0.05% to about 18%, about 0.05% to about 20%, about 0.05% to about 22%, about 0.05% to about 25%, about 0.05% to about 27%, about 0.05% to about 30%, about 0.05% to about 32%, about 0.05% to about 35%, about 0.1% to about 0.5%, about 0.1% to about 1%, about 0.1% to about 2%, about 0.1% to about 5%, about 0.1% to about 7%, about 0.1% to about 10%, about 0.1% to about 12%, about 0.1% to about 15%, about 0.1% to about 18%, about 0.1% to about 20%, about 0.1% to about 22%, about 0.1% to about 25%, about 0.1% to about 27%, about 0.1% to about 30%, about 0.1% to about 32%, about 0.1% to about 35%, about 0.5% to about 1%, about 0.5% to about 2%, about 0.5% to about 5%, about 0.5% to about 7%, about 0.5% to about 10%, about 0.5% to about 12%, about 0.5% to about 15%, about 0.5% to about 18%, about 0.5% to about 20%, about 0.5% to about 22%, about 0.5% to about 25%, about 0.5% to about 27%, about 0.5% to about 30%, about 0.5% to about 32%, about 0.5% to about 35%, about 1% to about 2%, about 1% to about 5%, about 1% to about 7%, about 1% to about 10%, about 1% to about 12%, about 1% to about 15%, about 1% to about 18%, about 1% to about 20%, about 1% to about 22%, about 1% to about 25%, about 1% to about 27%, about 1% to about 30%, about 1% to about 32%, about 1% to about 35%, about 2% to about 5%, about 2% to about 7%, about 2% to about 10%, about 2% to about 12%, about 2% to about 15%, about 2% to about 18%, about 2% to about 20%, about 2% to about 22%, about 2% to about 25%, about 2% to about 27%, about 2% to about 30%, about 2% to about 32%, about 2% to about 35%, about 5% to about 7%, about 5% to about 10%, about 5% to about 12%, about 5% to about 15%, about 5% to about 18%, about 5% to about 20%, about 5% to about 22%, about 5% to about 25%, about 5% to about 27%, about 5% to about 30%, about 5% to about 32%, about 5% to about 35%, about 7% to about 10%, about 7% to about 12%, about 7% to about 15%, about 7% to about 18%, about 7% to about 20%, about 7% to about 22%, about 7% to about 25%, about 7% to about 27%, about 7% to about 30%, about 7% to about 32%, about 7% to about 35%, about 10% to about 12%, about 10% to about 15%, about 10% to about 18%, about 10% to about 20%, about 10% to about 22%, about 10% to about 25%, about 10% to about 27%, about 10% to about 30%, about 10% to about 32%, about 10% to about 35%, about 12% to about 15%, about 12% to about 18%, about 12% to about 20%, about 12% to about 22%, about 12% to about 25%, about 12% to about 27%, about 12% to about 30%, about 12% to about 32%, about 12% to about 35%, about 15% to about 18%, about 15% to about 20%, about 15% to about 22%, about 15% to about 25%, about 15% to about 27%, about 15% to about 30%, about 15% to about 32%, about 15% to about 35%, about 18% to about 20%, about 18% to about 22%, about 18% to about 25%, about 18% to about 27%, about 18% to about 30%, about 18% to about 32%, 18% to about 35%, about 20% to about 22%, about 20% to about 25%, about 20% to about 27%, about 20% to about 30%, about 20% to about 32%, about 20% to about 35%, about 22% to about 25%, about 22% to about 27%, about 22% to about 30%, about 22% to about 32%, about 22% to about 35%, about 25% to about 27%, about 25% to about 30%, about 25% to about 32%, about 25% to about 35%, about 27% to about 30%, about 27% to about 32%, about 27% to about 35%, about 30% to about 32%, about 30% to about 35%, or about 32% to about 35% by weight of the palatable chewable composition. In some embodiments, the encapsulated primary ingredient may be from about 5% to about 10%, about 5.5% to about 9.5%, about 6% to about 9%, about 6.5% to about 8.5%, or about 7% to about 8% by weight of the palatable chewable composition.

In some embodiments, the primary ingredient may be acetaminophen. Acetaminophen may be present in an amount ranging from about 1% to about 20% by weight of the palatable chewable composition. The amount may be about from about 1% to about 2%, about 1% to about 4%, about 1% to about 5%, about 1% to about 6%, about 1% to about 8%, about 1% to about 9%, about 1% to about 10%, about 1% to about 11%, about 1% to about 12%, about 1% to about 13%, about 1% to about 14%, about 2% to about 15%, about 2% to about 16%, about 2% to about 18%, about 2% to about 19%, about 2% to about 20%, about 4% to about 5%, about 4% to about 6%, about 4% to about 8%, about 4% to about 10%, about 5% to about 16%, about 5% to about 18%, about 5% to about 20%, about 6% to about 18%, about 6% to about 20%, or from about 8% to about 10% by weight of the palatable chewable composition. The amount may be about 10%, about 11%, about 12%, about 13%, about 14%, about 15%, about 16%, about 17%, about 18%, about 19%, or about 20% by weight of the composition. The amount may be about from about 10% to about 12%, about 10% to about 14%, about 10% to about 15%, about 10% to about 16%, about 10% to about 18%, about 10% to about 20%, about 12% to about 14%, about 12% to about 15%, about 12% to about 16%, about 12% to about 18%, about 12% to about 20%, about 14% to about 15%, about 14% to about 16%, about 14% to about 18%, about 14% to about 20%, about 15% to about 16%, about 15% to about 18%, about 15% to about 20%, about 16% to about 18%, about 16% to about 20%, or from about 18% to about 20% by weight of the palatable chewable composition. The amount may be about 10%, about 11%, about 12%, about 13%, about 14%, about 15%, about 16%, about 17%, about 18%, about 19%, or about 20% by weight of the composition.

The primary ingredient may be in a solid or powder form. The primary ingredient may be further processed in grinding or attrition devices such as a cone mill, pin mill, disk refiner, granulator, powder mill, colloid mill, cone blender, and the like until a suitable particle size is achieved. A suitable particle size may be less than about 150 microns. In some embodiments, the Dv(90) may be less than 20% the size of the suitable particle size.

Suitable particle size ranges may be selected from the milled materials using appropriate techniques. For examples, the appropriate techniques may include sieving, wire sifting, aerodynamic sorting, centrifugal sorting, or a combination thereof. The particles may be entrained in an air or other fluid stream (including liquids and slurries) or otherwise conveyed into an encapsulating system to be given on or more coatings or other treatments. Suitable encapsulation methods include but are not limited to fluidized bed coating, spray drying, coacervation, in-situ polymerization, solvent evaporation, extrusion, hot melt extrusion, or electrospinning, and are discussed further below.

The encapsulated primary ingredient further includes an encapsulant. In the palatable chewable composition, an unpleasant flavor or aroma of one or more primary ingredients may be effectively masked without interfering with the function of the one or more primary ingredients. In particular, one or more materials may be applied to microencapsulate the one or more primary ingredients to mask unpleasant flavors or aromas and form the encapsulated primary ingredients. The tailored materials may form one or more coating layer(s). The coating layers may also protect the one or more primary ingredients from challenges faced during manufacturing and storage, while also providing, supporting, or being compatible with the intended release profile for the one or more primary ingredients.

The encapsulant may include lipids, waxes, polymers, proteins, synthetic or natural waxes, nanocarriers, misomes, hydrogels, chitosan, shellac, alginates, starch, polysaccharides, or other encapsulants and combinations thereof. Additional examples of encapsulants are described below, as well as suitable applications and purposes for the same.

In some embodiments, the palatable chewable composition product may include a flavor masking coating layer and a release regulating coating layer. In other embodiments, both the flavor masking coating layer and the release regulating coating layer may be the same material.

The release regulating coating layer may be adapted to control the release of the primary ingredient at a suitable location in the GI tract. The flavor masking coating layer may be adapted to provide flavor masking and/or in some aspects aroma masking. The flavor masking may persist in the mouth but begin dissolution or otherwise cease its barrier function in the stomach so that the release regulating coating layer will determine the release profile thereafter without substantial interference from the flavor masking coating layer. For example, to target the stomach, the active may be embedded in a swellable polymer that expands in the acidic environment of the stomach to increase release of the active and/or to promote floating in the gastric juices of the stomach to increase the retention of the compound in the stomach. For reaching the small intestine or colon, one approach is to encapsulate the active(s) in a pH sensitive material that does not readily dissolve or disintegrate or otherwise release the active above a pH of about 4.5, about 5.5, about 6.5, about 7, about 7.5, or about 8. Such coatings may include cellulose acetate trimellitiate (CAT) or hydroxypropylmethyl cellulose phthalate (HPMCP), polyvinyl acetate phthalate (PVAP), or shellac.

The layers may be composed of different materials depending on the primary ingredient. For example, the flavor masking coating layer may be a bitter masker, taste modulator, or high intensity sweetener embedded or mixed into a fat, wax, or ethylcellulose based coating layer. The release regulating coating layer may be a pH sensitive ingredient like Eudragit® or a moisture sensitive ingredient like a gelatin coacervation with diffusion rate-limited release.

The release regulating coating layer may be provided as the first layer applied to a primary ingredient, such that the materials of the release regulating coating layer are in contact with the primary ingredient or are at least closer to the primary ingredient than the flavor masking coating layer. In such aspects, the release regulating coating layer may be the primary coating layer on the primary ingredient with a further outer secondary coating layer, the flavor masking coating layer, which is applied to provide added masking efficacy beyond whatever masking may be provided by the release regulating coating layer.

In some embodiments, the flavor masking coating layer may be relatively closer to the primary ingredient and may be a primary coating layer that is in contact with the primary ingredient or at least closer to it than the more outward lying release regulating coating layer. The release regulating coating layer may be a secondary coating layer applied after the flavor masking coating layer. In some aspects, the flavor masking coating layer may be interdigitated or interspersed with the release regulating coating layer.

In some embodiments, a primary ingredient in or on a palatable chewable composition may have a multi-layer coating or multiple materials that provide various protective functions, such as a two, three, four, or more materials that may be coating layers. A multi-layer coating or multi-component treatment is adapted to control or provide multiple functions, with each material or layer providing a function such as flavor and aroma masking, regulating the release of the primary ingredient, protecting the primary ingredient from environmental factors (moisture, shear, pH, or temperature), preventing migration or diffusion of the primary ingredient into the palatable chewable composition, preventing migration of moisture or other ingredients in the palatable chewable composition into the primary ingredient, targeted delivery, enhancing stability, or other functions. The number of materials and the function of each layer or material may be adjusted based on the specific requirements of the application.

In some embodiments, the multiple layers may be concentric, with clear boundaries between the materials of each layer. In other embodiments, the materials may be interdigitated or interspersed for at least part of the coating thickness. This interdigitation or interspersed state need not be construed as miscibility, but generally rather as a physical intermingling of the distinct materials. Such layers may be applied at substantially the same time, as in a spray drying system or fluidized bed system with nozzle delivering droplets of both coating materials simultaneously, or with alternating bursts of differing materials being applied, or in multiple cycles as one coating material at a time is applied to build a lamellar or other complex structure.

In certain aspects, the distribution of the different coating materials on a primary ingredient may be distributed as an “island-sea” configuration. The “island-sea” configuration may include “islands” of a first material dispersed within a continuous body (“sea”) of a second material. Alternatively, other types of microstructures, such as co-continuous, layered, core-shell, or interpenetrating networks, may be formed based on the specific materials used and the method of blending.

Two or more of the coating layers may support similar functions. Functions include but are not limited to protecting against shear and fracture, preventing premature release, odor masking, and the like. In many aspects one layer will play a relatively more important role for one particular function such as regulating release of the primary ingredient or masking flavor or aroma, while a second layer may preferentially support a different second function.

The specific configuration (concentric vs. island-sea or other structure), composition, and thickness of each layer may be adjusted based on the desired release profile, the nature of the primary ingredient, and other application-specific requirements. Thickness itself may be difficult to measure accurately and difficult to control precisely, while the basis weight applied (e.g., grams of dry coating per gram of primary ingredient) is much easier to determine and control, and thus may be used on process control.

In some aspects, the first layer may be in direct contact with the encapsulated primary ingredient and may serve to regulate the release of the primary ingredient. This layer may be composed of a specific material or combination of materials known for their controlled-release properties. In another aspect, the first layer may not directly contact the encapsulated active because it may be already embedded in another material. Materials that the primary ingredient may be embedded in include but are not limited to lipids, gelled materials, viscous slurry, cream, and the like.

In other aspects, the first layer may be a flavor masking coating layer that prevents the primary ingredient from influencing the flavor experienced during ingestion. This layer may also reduce migration of the primary ingredient into the matrix of the palatable chewable composition during storage and likewise inhibit migration of other agents in the palatable chewable composition matrix into the primary ingredient, including moisture. In such aspects, the release regulating coating layer may be provided as a secondary layer closer to or including the outer surface of the encapsulated primary ingredient.

The multi-layer coating may generally provide enhanced stability that may be jeopardized by the manufacturing process, including the properties of the solution or solutions used to produce the matrix or by long-term contact with the matrix of the palatable chewable composition. The palatable chewable composition matrix is typically made from a heated aqueous solution having gelling agents, sweeteners, flavorings, pH control agents, and the moisture from the solution or the gelled matrix may be harmful to many actives, as can the pH range and other aspects of the materials in contact with encapsulated primary ingredients. Further, the elevated temperature of the aqueous solution when the actives are blended in may also be harmful to enteric coatings on the actives or otherwise damage the primary ingredient. Furthermore, high shear during blending may damage the primary ingredient or its coating. But with the methods, compositions, and processes described herein, palatable chewable compositions may be provided in which the actives and their coatings are protected from the manufacturing process and achieve higher stability than is possible with conventional approaches, resulting in palatable chewable compositions that are more efficacious in delivering one or more actives as intended to provide benefits to the user, while still also having the desired masking benefit relative to one or more unpalatable flavors or malodors.

In one aspect, the palatable chewable composition matrix holds one or more primary ingredients that may be coated with one or more coatings to form one or more encapsulated primary ingredients. In some embodiments, the palatable chewable composition matrix may include a first coating for a first primary ingredient and a second coating for a second primary ingredient. As stated above, the first and second coatings may increase the stability of the primary ingredients during exposure to manufacturing conditions and during prolonged storage after manufacturing. For example, the coating may provide both a flavor or odor masking benefit and also a protective function to the one or more primary ingredients.

The coating may provide protection to the primary ingredient from relatively high water content in the palatable chewable composition. For example, the water content may be from about 5% to about 30%, about 6% to about 29%, about 7% to about 28%, about 8% to about 27%, about 9% to about 26%, about 9% to about 25%, about 10% to about 24%, about 11% to about 23%, about 12% to about 22%, about 13% to about 21%, about 14% to about 20%, about 15% to about 19%, or about 16% to about 18% by weight of the palatable chewable composition.

The coating may provide protection to the primary ingredient from relatively high water activity level. For example, the water activity level may range from about 0.4 to about 0.9, about 0.5 to about 0.8, or about 0.6 to about 0.7.

The coating may provide protection to the primary ingredient from acidic or alkaline pH. Acidic pH includes a pH less than about 6.5, such as about 6.0, about 5.5, about 5.0, about 4.5, about 4.0, about 3.5, or about 3.0. Alkaline pH includes a pH above about 7.0, such as about 7.5, about 8.0, about 8.5, about 9.0, about 9.5, about 10.0, about 10.5, about 11.0, or about 11.5.

The coating may provide protection to the primary ingredient from oxidation related to the oxygen level experienced by the primary ingredient during manufacturing and storage.

The coating may provide protection to the primary ingredient from elevated temperature. For example, the coating may provide protection to the primary ingredient from temperatures ranging from about 40° C. to about 150° C., about 50° C. to about 150° C., about 60° C. to about 150° C., about 70° C. to about 150° C., about 80° C. to about 150° C., about 40° C. to about 50° C., about 50° C. to about 60° C., about 90° C. to about 140° C., about 100° C. to about 130° C., or about 110° C. to about 120° C.

The coatings may provide protection to the primary ingredient from elevated shear or applied power density during manufacturing and from prolonged exposure to the matrix during the shelf life of the palatable chewable composition; wherein the one or more coatings are also effective in providing a suitable release profile to the user for the one or more primary ingredients after ingestion. The different release profiles may be instant or immediate release, sustained or extended release, and delayed release. The release profile is affected by the drug and/or drug formulation to be administered (e.g., coatings, specific excipients, and other drug product attributes), the concentration of drug in the formulation, the medium for which the release or dissolution of the product takes place, and a variety of other factors that are appreciated by those of ordinary skill in the art. A suitable release profile may include an immediate release profile, an extended release profile, or a delayed release profile.

In some aspects, the protective function for each of the one or more coatings may be protecting the primary ingredients or any internal coating layers from any one of: (1) a maximum exposed temperature Tₘₐₓ encountered during blending of the one or more one primary ingredients with the aqueous phase, (2) a maximum shear level τₘₐₓ experienced during blending, (3) the moisture level of the aqueous phase or maximum activity level aₘₐₓ of the water in the aqueous phase during manufacturing and/or during shelf life (storage after manufacturing), (4) adverse reactions with other ingredients in the palatable chewable composition, (5) oxidation, and/or (6) unwanted damage to (e.g., etching, erosion, fracture, or dissolution of) a coating layer intended to control release of the active in the GI tract. Further, any one or more of the coatings may also provide a masking function relative to the odor or flavor of a primary ingredient.

In some aspects, the encapsulated primary ingredients may have two or more coating formulations, such as two or more types of outer coatings, that provide two or more different protective functions, or different degrees of protection relative to the various challenges faced during manufacturing and storage. For example, a first primary ingredient may have a coating designed to resist elevated shear (e.g., to protect internal materials that are fragile and might break or be released prematurely when subject to the shear stresses of mixing) more effectively than the coatings on other primary ingredients also present, while a second primary ingredient may have a coating designed to resist elevated moisture (e.g., to protect a moisture-sensitive internal coating for extended release once in the stomach or intestines) relatively more effectively (or to resist a higher moisture level or water activity) than other coated primary ingredients in the palatable chewable composition product, and a third primary ingredient may have a coating designed to resist elevated temperature (e.g., a shellac or other polymer) relatively more effectively than other coated primary ingredients in the palatable chewable composition product in order to protect internal lipids that otherwise would melt and be dispersed during manufacture. Examples of coatings include but are not limited to crosslinked hydrocolloids, modified starches and celluloses, waxes, or shellac.

In some aspects, a single primary ingredient may be segregated into two or more groups of particles that were treated in two or more coating processes with different doses and/or different coating materials and/or different coating and drying methods, such that a first portion of the primary ingredient is provided with a coating adapted to resist a first challenge such as elevated temperature, shear, or moisture level, while a second portion of the primary ingredient is provided with a different coating (different in terms of chemical composition or other physical properties such as thickness, porosity, hardness, or elasticity) adapted to resist a second challenge. In some aspects the second challenge is different from the first challenge, such as the first challenge being elevated shear or temperature while the second challenge may be elevated moisture. A single primary ingredient may be partitioned into any number of subsets that each are independently encapsulated with various coatings, including various multilayer coatings, and any number of primary ingredients may be present in the palatable chewable composition and may receive independent coatings to provide an array of encapsulants adapted to resist a variety of challenges.

In some aspects, a palatable chewable composition is provided having one or more encapsulated primary ingredients in contact with a matrix having water, a sweetener, and a hydrocolloid. The one or more encapsulated primary ingredients may include a first encapsulated primary ingredient comprising a first coating material adapted to withstand a first challenge during manufacturing and storage and a second encapsulated primary ingredient comprising a second coating material adapted to withstand a second challenge during manufacturing and storage. The first and second challenges are moisture level, water activity, elevated temperature, elevated shear, a pH range, adverse reactions with other ingredients in the palatable chewable composition, oxidation, etching, erosion, fracture, or dissolution. One or more of the first and second challenges may adversely affect product stability, such as the stability of one or more of the encapsulated primary ingredients or the stability of one or more coating layers on at least one of the encapsulated primary ingredients. The first and second challenge may be different in some aspects or may be the same or similar in other aspects. A coating material may be effective against multiple challenges but may be relatively more effective against a particular challenge relative to one or more other coatings serving as encapsulants in the palatable chewable composition. For example, high shear may jeopardize a water repellant layer protecting a first primary ingredient from moisture, such that high shear may jeopardize the stability of the first primary ingredient by allowing moisture from the matrix solution or the gelled matrix to reach the first primary ingredient and decrease its shelf life or cause premature release of the active, such that the intended release profile (e.g., extended or delayed release in a target region of the GI tract) is not achieved after ingestion, or that other functions of the encapsulant such as flavor masking or reduction of oxidation are not adequately achieved.

In one aspect, a palatable chewable composition is described having a first primary ingredient with a primary coating such as an enteric coating designed to reach a targeted region of the GI tract in order to deliver the primary ingredient at a suitable region therein (e.g., probiotics to the small intestines and colon, iron to the stomach, a bitter agent coated in order to mask the flavor while in the mouth), while also being coated with a suitable secondary coating to improve the stability of the first primary ingredient. Such stability benefits may be achieved by providing protection relative to the environment manufacturing (e.g., blending particles of active material into the heated aqueous solution that is used to produce the matrix under conditions of relatively high water activity, high temperature, a potentially incompatible pH range, and elevated shear) and the long-term storage of the product throughout its shelf life. The secondary coating may be made from a different material than the primary coating and may be, for example, a wax, a shellac, a water-resistant or shear-resistant polymer. A second primary ingredient (or any number of additional primary ingredients) may also be present and may also be encapsulated, again with one or more coating layers that are suitable for the environments encountered during manufacturing and thereafter during transportation and storage throughout the product's shelf life.

In one aspect, one or more coating layers as described herein may be applied to the matrix of the palatable chewable composition. For example, one or more coating layers on an encapsulated primary ingredient in or on the matrix of a palatable chewable composition is designed to prevent migration of water into the active, while also limiting the migration of the active into the water of the matrix or into the matrix itself. Maintaining the active in a relatively dry state segregated from the matrix during storage and from the aqueous solution used to create the matrix during manufacturing may be achieved to various degrees with the methods, processes, and compositions described herein. Such “waterproofing,” though believed to be rarely considered in prior work, has been found in our investigations to be an useful consideration. In some aspects, a water-resistant coating is provided as a layer of an encapsulated particle of an active. The water-resistant coating may be adapted to withstand the moisture level or water activity of the aqueous solution during manufacturing and also to withstand the moisture level or water activity of the resulting matrix after manufacturing and during storage over the shelf life of the palatable chewable composition product.

In one aspect, the stability of a primary ingredient in a palatable chewable composition matrix may be enhanced by coating the primary ingredient with a moisture resistant material that prevents direct contact with an aqueous matrix solution during manufacturing and resists migration of water into the primary ingredient during storage while the primary ingredient is in the matrix of a palatable chewable composition. Such water-resistant materials may include shellac, such as shellac that is also coated with or combined with a hydrophobic material such as a wax, a polyolefin such as polyethylene or polypropylene. Also to be considered are acrylate polymers (e.g., poly-ethylacrylate, poly-methylmethacrylate), Eudragit coatings, ethylcellulose, polyolefins, zein, waxes and fatty acids, latexes such as those made from ethylcellulose and related polymers, combinations of nanocellulose with any of the polymers mentioned herein, including coated nanocellulose, methacrylate aminoester copolymers, methacrylate ester copolymers. Such coatings may also be associated with, contain, or be combined with oxygen scavengers, antioxidants, swellable materials such as hydrogels that can swell in response to high moisture content to preclude further migration of moisture through the hydrogel, further hydrophobic agents, or crosslinkers.

In another aspect, a palatable chewable composition is provided with a matrix having two or more separately encapsulated primary ingredients embedded or dispersed therein, wherein each primary ingredient is provided with one or more coating layers that are collectively adapted to both protect the primary ingredient during manufacturing as well as to provide an intended release profile for the primary ingredient in the human body, such as releasing the primary ingredient in the proper region(s) of the GI tract after ingestion to deliver an intended physiological or other benefit to the user. A coating for protection of the primary ingredient during manufacturing may include a protective layer for protecting a release control layer that controls release of the primary ingredient such that through the protection provided, the release control layer is not compromised by shear, elevated temperature, or the activity of water during manufacturing and subsequent storage, thus keeping the release control layer or associated materials intact so that the desired release profile may be achieved. Thus, the palatable chewable composition product may include a first primary ingredient encapsulated in a first coating and a second primary ingredient encapsulated in a second coating. The first coating and the second coating may be adapted to independently provide delivery of the first ingredient and delivery of a second to a targeted location in the GI tract, and may also be adapted to provide a release profile having one or more of extended release, delayed release, immediate release, pulsatile release, biphasic or multiphasic release, responsive release (e.g., responsive to a pH), and chronotherapeutic release. The second coating may also be adapted to result in a specific release profile that may be different from the release profile of the first coating, such that the functions of the first and second coating may be independently controlled and delivered through the independent release characteristics of the respective coatings relative to their respective ingredients.

Various polymers may be used to construct a coating for encapsulation of primary ingredients. For coatings adapted to provide masking effect relative to a flavor or odor that may be unpleasant, polyacrylate-based polymers such as Eudragit® materials may be used. For example, Eudragit ER is a cationic polymer based on dimethylaminoethyl methacrylate, butyl methacrylate, and methyl methacrylate. It is soluble in gastric fluid as well as in weakly acidic buffer solutions, up to pH~5. When serving as an outer layer in microencapsulation, it forms swellable, permeable, and insoluble films at pH 5 or higher (as in saliva which has a pH from about 6.7-7.6), yet dissolves rapidly by forming salts at a pH less than 5 (as in gastric fluids which have a pH from about 1.0 to 1.7).

Hydroxypropyl methylcellulose (HPMC) may also be used as a coating material. HPMC may form a gel-like matrix that can help control the release of the primary ingredient in the GI tract while also reducing the risk of unpleasant flavors or odors. Polyvinyl alcohol (PVA) may be used for its film forming capabilities that can control the release of the primary ingredient while also being able to plasticize several film-forming encapsulants. Alginate such as sodium alginate or calcium alginate is a natural polymer with potential to control the release of some primary ingredients and help mask flavors. Gelatin may also be used in masking flavor and can also aid in controlled release of a primary ingredient, as can chitosan, a natural polymer derived from chitin. Film-forming salts of chitosan (e.g., chloride, lactate or gluconate) may also be considered for flavor and aroma masking.

Such coatings may be applied in one or multiple layers, and may include lipids such as glycerides and waxes that may have a melting point or melting range of at least 20° C. Lipids with a melting point from about 20° C. to about 150° C., about 30° C. to about 140° C., about 40° C. to about 130° C., about 50° C. to about 120° C., about 60° C. to about 110° C., about 70° C. to about 100° C., or 80° C. to about 90° C. may also be used. In some aspects, lipids with a melting point or range such that the coating will soften in the mouth may be used. Such waxes, for example, may include synthetic and natural waxes such as animal waxes, vegetable waxes, petroleum waxes, beeswax, lanolin, bayberry, candelilla, sunflower, carnauba, paraffin, microcrystalline petrolatum, carbowax alone or in mixtures. Alternatively or in addition, the coatings may include various polymers such as proteins, polypeptides/poly-amino acids (e.g. polyglutamic acid, polyaspartic acid, polylysine), polyacrylamide, poly N-acylhydroxypropine esters, polylactic acid, polyglycolic acid, polylactic-co-glycolic acid, polysebacic acid, polyfumaric acid, polymers formed from hydroxyethylmethacrylate, polymers formed from ethylene bismethacrylate, carboxymethylcellulose and its derivatives, gum arabic, agarose, alginate such as sodium or potassium alginate, polyphosphate, heparin, gelatin, guaran, copolymers of sebacic acid and fumaric acid, copolymers of biscarboxyphenoxypropane and sebacic acid, poly(carboxyphenoxyacetic) acid, poly(carboxyphenoxyvaleric) acid, poly-ε-caprolactone and related polyesters, poly-ε-caprolactone-co-δ-valerolactone, poly-ε-caprolactone-co-lactic acid, hyaluronic acid, chitin, chitosan, dextran, dextrin, carboxy-dextran, diethylaminoethyl dextran, aminoethyl dextran and dextran sulfate, collagen, albumin, fibrinogen, and other naturally occurring polymers, such as collagen, starch, amylose, carboxymethyl amylose, hydroxyethyl amylose, hydroxyethyl amylose, cellulose, carboxylmethyl cellulose, ethyl cellulose, hydroxyethyl cellulose; agarose, pectic acid, alginic acid, gum arabic, galactomannan, levan, hyaluronic acid, polyalkylene glycols such as polyethylene glycol, polypropylene glycol, polybutylene glycol, or mixtures thereof.

Synthetic and natural waxes that may be useful as coatings for the primary ingredients include animal waxes, vegetable waxes, petroleum waxes, and the like. Specific examples of waxes are beeswax, lanolin, bayberry, candelilla, carnauba, paraffin, microcrystalline petrolatum, or carbowax. Mixtures of these waxes are also useful.

Encapsulants may also include nanocarriers. Nanocarriers may include nano-delivery systems that carry drugs on the nanoscale and may provide improved drug stability, controlled release, or targeted delivery. Such nanocarriers may include: (1) Vesicular systems that involve vesicles, or small, enclosed sacs that can carry drugs, which may include liposomes, niosomes, and catanionic vesicles, for example; (2) Colloidal drug delivery systems, such are mixtures in which one substance is dispersed throughout another at a colloidal scale, which may include liposomes, niosomes, micelles, and the like; (3) Bilayer systems in which a bilayer of lipids or surfactants that form a sphere that can enclose an active; (4) Self-assembled structures such as micelles and vesicles, that are sometimes referred to as self-assembled or self-organizing systems; (5) Surfactant-based systems, which refers to systems that utilize surfactants to improve drug delivery. These may include micelles, niosomes, and catanionic vesicles; and (6) Lipid-based delivery systems may, including but not limited to include liposomes and solid lipid nanoparticles and nanostructured lipid carriers. Liposomes may be created from cholesterol and natural non-toxic phospholipids to encapsulate both hydrophilic and lipophilic drugs, protect the drug from degradation, and control the release of the drug. Depending on their design, liposomes can potentially deliver a drug to various parts of the gastrointestinal (GI) tract, not just the stomach. Niosomes likewise are vesicles employing non-ionic surfactant vesicles that, like liposomes, can encapsulate both hydrophilic and lipophilic drugs. Related structures employing cationic and anionic surfactants may be considered, including catanionic vesicles. The surface charge of cationic vesicles can also be tuned by adjusting the ratio of cationic to anionic surfactant, which can influence the vesicles' interaction with biological membranes and their release behavior.

Micelles may also be used for drug delivery. Unlike vesicles, which have an aqueous core surrounded by a lipid bilayer, micelles have a hydrophobic core surrounded by a hydrophilic shell. This makes them suitable for the encapsulation and delivery of lipophilic drugs.

By way of example, encapsulation may also be achieved by coating an active with pectin, agar, carrageenan, alginate, and other polysaccharides or polymers, including chitosan, sodium alginate and other alginate salts, xanthan, curdlan, guar gum and other polysaccharides (particularly ionically as well crosslinkable polysaccharides), as Eudragit (polymethylmethacrylate polymers), as encapsulating agents.

Hydrogel coatings on primary ingredients may be used to enhance control of the release of primary ingredients and enhance the stability of the primary ingredients. For example, hydrogels can swell when in contact with an aqueous environment and thereby modify the diffusion paths for the active, and stimuli-responsive hydrogels in particular such as poly(methacrylic acid) nanogels can swell in response to a change in conditions such as a rise in pH, which can make them useful in controlled release applications. Initiated chemical vapor deposition may be used to form a film that can encase primary ingredients in the hydrogel coating without the use of a solvent that might threaten the stability of the active. Such material may be adapted to prevent diffusion of an active when in a low pH environment such as palatable chewable composition as described herein, but when the pH increases such as in the GI tract after the stomach, the hydrogel can change to allow greatly increased diffusion. A crosslinker may be varied in concentration in order to adjust the hydrogel performance.

In some aspects, a hydrogel material may be used for components of the palatable chewable composition such as an encapsulated complex encasing one or more primary ingredients with a secondary matrix within the complex surrounding the primary ingredients. Primary ingredients may be encapsulated with or otherwise combined with one or more hydrogels via a wide variety of methods, including spray drying and vapor-phase deposition methods such as initiated chemical vapor deposition (iCVD), plasma enhanced chemical vapor deposition (PE-CVD), and atomic layer deposition (ALD).

Chitosan, alginates such as sodium alginate, xanthan, curdlan, guar gum and other polysaccharides (particularly ionically crosslinkable polysaccharides) may be considered. Cellulose acetate phthalate (CAP) and other cellulose derivatives may be considered, as well as cellulose acetate trimellitate (CAT), polyvinyl acetate phthalate (PVAP), polyvinylacetate phthalate, HPMCP, polyethylene glycols, and the like.

By way of example, starch and other polysaccharides may be considered for a wide variety of applications. Starches may be fabricated into porous starch, microgels, molecular aggregates, starch granule aggregates, and other forms of various porosity, diffusivity, solubility, and enzymatic resistance, properties that may be adapted to enhance the stability of one or more primary ingredients. Such systems may be used to encapsulate, protect, and deliver many primary ingredients. Starch-based coatings may be applied by spray drying, extrusion, freeze drying, sonication using ultrasound or ultrasonic homogenization, ultra-high pressure, coagulation, fluidized bed coating, molecular inclusion, chemical, and enzymic methods. For example, a starch combined with oleic acid or other lipids may be combined and coated onto a particle to provide a delayed release of controlled release mechanism. In general, controlled release of primary ingredients may be achieved with starch-based microcapsules (or nanocapsules) that encapsulate the primary ingredients. Such encapsulants can help provide bioavailability, resistance to enzymes, protection of probiotics, masking of bitterness or other bad flavors, and may be tailored to reach the small intestines, colon, and/or stomach. A variety of starch-based microcapsule wall materials may be considered, such as cyclodextrins and related crown compounds, amylose, octenylsuccinic anhydride (OSA)-modified starch, and numerous other modified starches such as cationic starch, anionic starch, oxidized starch, cross-linked starch, acid-hydrolyzed starch, amylolytic starch, debranching starch. Starches combined with lipids may be considered for a variety of water-resistant layers.

Starch-based microcapsule delivery systems often employ starch that has been modified by physical, chemical or enzymatic methods. Such physical methods may include (1) microwave treatment which may be carried out with the starch in the presence of lipids such as linoleic acid, oleic acid, stearic acid, ethyl cis-9-octadecenoate, ethyl cis, cis-9,12-octadecadienoate and methyl octadecenoate; (2) high pressure homogenization, wherein high shear, impact and cavitation can break down starch granules as high-pressure liquid passes the gaps of a homogenizer valve, resulting in dissolving of amylose that can then be compounded with other molecules to form a complex; (3) ultrasonic treatment, in which the cavitation produced by ultrasonic waves in water can result in high shear that can break down starch granules and assist in preparing a starch-based encapsulant; (4) ultra-high pressure treatment that can modify hydrogen bonding in starch and lead to improved combinations with other materials resulting in hydrogels, for example; (5) high-speed shearing, and (6) freeze drying.

Chemical methods for modifying starch may include (1) esterification that may, for example, be used to provide OSA-modified starch, which may further be combined with gum Arabic or other agents to yield a useful encapsulant mixture; (2) oxidation; (3) acid hydrolysis; and (4) cross-linking (e.g., sodium trimetaphosphate/sodiumtripolyphosphate cross-linking, which may be done, for example at elevated pH such as 10 or higher or 11 or higher in the presence of sodium sulfate).

Enzymatic methods may include production of amylolytic starch using enzymes such as α-amylase, β-amylase, and glucosidase, which may be useful in producing porous starch. Debranching starch can also be produced, such as through treatment of starch usually treated with starch debranching enzymes such as pullulanase and isoamylase. Cyclodextrin containing 6-12 D-glucopyranose units may also be considered a type of enzymatically modified starch since it is a cyclic oligosaccharide that may be produced from amylose in the presence of cyclodextrin glucosyltransferase found in Bacillus bacteria.

Modified starches can also include cationic and anionic starches, whose charge characteristics may be helpful in encapsulating specific compounds. Also to be considered is resistant starch. Resistant starch generally can resist the hydrolysis from the enzymes in the stomach and the duodenum and can then be degraded by microbiota in the colon. Resistant starch has four broad classes, Types 1, 2, 3, and 4. Type 1 may be naturally occurring starches that resist digestion, including those found in legumes and milled grains. Type 2 has non-gelatinized granules that may be slowly digested in the small intestine but largely reach the large intestine and are found in green bananas, raw potatoes, or high amylose corn. Type 3 is cooked in water until fully hydrated and is then cooled. Type 4 includes chemically modified starches that may be difficult to digest.

For delivery of a primary ingredient after the stomach, various enteric coatings may be considered that can withstand the low pH in the stomach and then may release their encapsulated primary ingredients in the following higher pH region of the small or large intestines. Such materials may include shellac, zein, methyl acrylate-methacrylic acid copolymers, cellulose acetate phthalate (CAP), hydroxypropyl methyl cellulose phthalate, hydroxypropyl methyl cellulose acetate succinate, cellulose acetate succinate, sodium alginate, polyvinyl acetate phthalate (PVAP), methyl methacrylate-methacrylic acid copolymers, cellulose acetate trimellitate, and the like.

Shellac in various forms may be useful in enhancing stability of various primary ingredients and may, for example, improve the water-proofing of a primary ingredient or protect lipids and other agents to collectively improve the stability of the encapsulated primary ingredient during manufacturing and storage. Shellac may be provided in solution form such as in ethanol or other solvents, including alkaline aqueous solutions such as those comprising ammonia or ammonium salts in which the solvent(s) can then be removed in spray drying or drying with fluidized bed application or application by other means. When ammonium salts were used to prepare the shellac solution, after drying the salts can subsequently be heated to release ammonia as a gas that can then be separated from the encapsulated primary ingredient.

The shellac may be neat shellac or shellac in combination with other materials apart from the solvent, such as alginate (e.g., alginate with calcium ions), whey protein or other proteins such as zein, cellulosic or other polysaccharide materials including nanofibers such as nano cellulose, nanospun fibers. Such added materials can affect the porosity, diffusivity, or pH sensitivity, of the shellac to best fit a desired release profile for an active. In some cases, an agent such as sorbic acid is combined with shellac that can promote disintegration when in the GI tract. The ratio of sorbic acid or other additive to the shellac on a solvent-free basis may range be from about 0.3% to about 15%, from about 0.5% to about 10%, or from about 1% to about 8% by weight. Similar principles may apply for any additives relative to dry coating materials for primary, secondary, or other coatings.

Shellac may be used over one or more coating layers that are susceptible to elevated temperature to hold them in place until cooled. Thus, a lipid layer may comprise a first or second layer on an active and a shellac layer may be part of an outer layer that helps protect the internal materials when at elevated temperature, such that they are held in place rather than separating from the active material when molten in a relatively high-temperature matrix solution.

Shellac tends to be a weak acid that has a pH-dependent dissolution profile. Dissolution tends to be low at low pH, where it normally may be effective in maintaining integrity and preventing release of primary ingredients in stomach acids. Dissolution may proceed more rapidly at a pH of about 7, such as at 7.3 or above. In some embodiments, shellac is selected that has a predetermined acid value, such as an acid value of at least 60, 65, 70, or 75. Higher acid values may be useful in some aspects when increased stability during storage is needed or increased resistance to stomach acids is desired. However, in some aspects, a lower acid value level may be useful in achieving a desired release profile or in terms of computability with the materials it contacts. Thus, in some aspects, a shellac is applied as a coating layer for a primary ingredient or other portions of a palatable chewable composition, including an outer layer of the entire palatable chewable composition or an amount of a gelled material, wherein the acid value is less than 70, 65, 60, 55, or 50, or from 40 to 70, 40 to 65, 40 to 60, 40 to 55, or 40 to 50. Acid value is measured according to the European Pharmacopoeia (Ph. Eur.), with ground shellac dissolved in a 1:1 mixture of diethyl ether and ethanol and titrated with 0.1 M potassium hydroxide solution.

Whether shellac solution is applied in a water-ethanol solution, an alkaline (e.g., ammonia) solution, or in another solvent system, it may be combined with additional agents that may acts as pore formers, plasticizers, or strength modifiers, including, for example, one or more of hydroxypropyl methylcellulose (HPMC), triethyl citrate (TEC), adipic acid, alginic acid, benzoic acid, citric acid, fumaric acid, sorbic acid, gelatin such as gelatin type B, agar, gums such as xanthan gum, lactose, proteins such as zein or casein, salts of any of the aforementioned acids such as sodium citrate or sodium alginate, a starch, cyclodextrin, maltodextrose, and the like.

The encapsulant may further include one or more plasticizers and/or other additives. The addition of plasticizer may reduce the risk of pinholes or breaks in the coating and provide a more uniform coating. Suitable plasticizers include but are not limited to polyols, organic esters, or oils/glycerides. The plasticizers may be low volatility materials with average molecular weights between 200 g/mol and 600 g/mol or from 200 g/mol to 400 g/mol. Examples of low volatility materials include but are not limited to diesters derived from dicarboxylic acids (e.g., sebacic acid, azelaic acid) or from ethylene glycol and propylene glycol, citric acid (e.g., tributylcitrate), or glycerol (e.g., triacetin or glycerol triacetate, tributyrin). Other examples of plasticizers include glycols and polyethylene glycols (e.g. PEG 400), sugar alcohols (e.g. sorbitol, erythritol, mannitol, xylitol, fructose, sucrose), triethyl citrate, acetyl tributyl citrate (ATBC), ethyl acetate, methyl acetate, ethyl formate, isopropyl acetate, esters (e.g. anisole, tert-butylmethyl ether, ethyl ether), dimethyl sulfoxide, alcohols (e.g. 2-methyl-1-propanol, 1- or 2-butanol, or 1-pentanol); or sorbitol sorbitan solution.

Plasticizers may be hydrophobic or hydrophilic. The hydrophilicity or hydrophobicity may be useful when preparing barrier materials for use in encapsulation. Examples of hydrophilic materials include but are not limited to glycerin, polyethylene glycols, polyethylene glycol monomethyl ether, propylene glycol, sorbitol sorbitan solution. Examples of hydrophobic plasticizers include acetyl tributyl citrate, acetyl triethyl citrate, castor oil, diacetylated monoglycerides, dibutyl sebacate, diethyl phthalate, triacetin, tributyl citrate, triethyl citrate, or other suitable citric acid esters.

Plasticizer efficacy depends on the coating materials used. For example, sugar alcohols alone or in combination with glycerol may be effective with starch-based films as well as polysaccharides such as pullulan-alginate-carboxymethylcellulose systems, while other compounds such as triacetin may be useful in plasticizing hydrogels and various polymers. Chitosan and its film-forming salts (e.g., chloride, lactate or gluconate) may be plasticized by glycerol.

Other additives may include pigments, clays, filler particles, nanocellulose crystals and microfibrillated cellulose, buffering agents and acidulants (e.g., citric acid, malic acid, and tartaric acid), rheology modifiers, detackifiers, preservatives such as potassium sorbate and sodium benzoate (which may also serve as sources of porosity in some coatings for control over release properties), humectants such as glycerin and sorbitol, glazing agents such as beeswax and carnauba wax; emulsifiers such as lecithin, oleates and polysorbates; stabilizers such as pectin and carrageenan to maintain the texture and consistency of the palatable chewable composition (though these may also be part or all of the gelling agents used in a particular matrix recipe), or anti-sticking agents such as mineral oil and cornstarch.

Microencapsulation methods include chemical polymerization, physicochemical microencapsulation (e.g., using a film-forming composition to encase the primary ingredient), and mechanical microencapsulation. Chemical polymerization involves a monomer in liquid that polymerizes at the interface of a primary ingredient to encapsulate it. This can occur via coacervation. In coacervation, the solubility of a polymer is decreased by addition of a salt or other material, or a change in pH and/or temperature. The change causes the polymer to precipitate on and around particles of the primary ingredient. Another approach is ionotropic gelation in which a polymer solution also containing the primary ingredient is formed into droplets which are added to a solution containing ions that can cross-link the biopolymer, causing it to form a gel.

Mechanical encapsulation typically involves applying a liquid or slurry of a coating material to particles of the primary ingredient and then removing a solvent to leave a solid or gelled coating on the active. This may be done using fluidized bed coating, solvent evaporation/extraction, or spray drying.

The microencapsulation coating may generally provide enhanced stability to the active that otherwise would be jeopardized by the manufacturing process, including the properties of the solution or solutions used to produce the matrix or by long-term contact with the matrix of the palatable chewable composition. The palatable chewable composition matrix is typically made from a heated aqueous solution having gelling agents, sweeteners, flavorings, or pH control agents, and the moisture from the solution or the gelled matrix may be harmful to many primary ingredients, as can the pH range and other aspects of the materials in contact with encapsulated primary ingredients. Further, the elevated temperature of the aqueous solution when the primary ingredients are blended in may also be harmful to enteric coatings on the primary ingredients or otherwise damage the active. Further still, high shear during blending may damage the active or its coating. But with the methods, compositions, and processes described herein, palatable chewable compositions may be provided in which the primary ingredients and their coatings are protected from the manufacturing process and achieve higher stability than is possible with conventional approaches, resulting in palatable chewable compositions that are more efficacious in delivering one or more primary ingredients as intended to provide benefits to the user, while still also having the desired making benefit relative to one or more unpalatable flavors or malodors. Suitable encapsulation methods include but are not limited to fluidized bed coating, spray drying, coacervation, in-situ polymerization, solvent evaporation, extrusion, hot melt extrusion, or electrospinning.

Fluidized bed coating may include Wurster coating or fluid bed microencapsulation, in which particles of the primary ingredient in a fluidized bed are carried by an air flow to create a cyclic movement of material with one or more spray nozzles at the bottom of the fluidized bed of particles (or elsewhere), coating particles with an atomized material to create a core-shell structure.

When spray drying is used, a solution or suspension of the primary ingredient and a coating material is sprayed into a hot chamber. The solvent evaporates, leaving behind small particles of the primary ingredient coated with the material. The resulting powder can then be incorporated into the palatable chewable composition. Spray drying may include partly drying a composition in a spray drying tower, wherein the composition includes a suspension and an emulsion of a primary ingredient and an aqueous solution of a coating material at an air temperature of about 70° C. to 200° C., or from 90° C. to 180° C., or from 110° C. to 160° C., to form partly dried particles (e.g., having a semi-solid skin), and further drying the particles at an air temperature between 0° C. to 70° C. such as from 30° C. to 60° C. to yield substantially dry particles with coating layers.

Coacervation may include the separation of a solution into two liquid phases, one of which forms a coating around the primary ingredient particles. The coating is then hardened to form the microcapsules.

In in-situ polymerization, a primary ingredient may be coated with a coating material that polymerizes directly on the surface of the primary ingredient (e.g., particles). Polymerization may be driven by UV light, by an initiator such as a peroxide (e.g., a hydrogen peroxide spray in the fluidized air stream as the products are being treated or transported), or by temperature.

In solvent evaporation, the primary ingredient and a coating material are dissolved in a common solvent, which is then evaporated to form microcapsules.

Extrusion may be used in processes such as alginate encapsulation, which is commonly achieved by nozzle extrusion and external gelation. Using this method, primary ingredient-containing alginate droplets formed at the tip of nozzles fall into a solution containing divalent cations that cause ionotropic alginate gelation as they diffuse into the droplets. Alginate may be used alone or on combination with other agents such as gelatin, pigment particles, silica materials, cyclodextrin, a shellac coating, to improve its flavor masking capability or for other effects. The use of cyclodextrin, for example, may assist in both flavor and odor masking.

Hot melt extrusion (HME) may be used to encapsulate primary ingredients within a meltable binder. The binder is generally a pharmaceutically acceptable meltable material that may include methyl and ethyl methacrylate anionic based copolymer (e.g., Eudragit L100 and Eudragit L100-55), polyethylene glycol (PEG), polyvinylpyrrolidone (PVP), and hydroxypropyl methylcellulose (HPMC), or lipids such as waxes, solid or semi-solid fats, glyceryl monostearate or glyceryl behenate. The primary ingredient may be any pharmaceutical compound that is stable under the processing conditions and may be dispersed within the binder. A plasticizer may enhance the processing and in turn result in better taste masking performance. In some cases, ionic interaction between the meltable material and the primary ingredient can lead to enhanced taste suppression. The extruder may be a twin-screw extruder or other types such as gear pump extruders, multi-screw extruders, single-screw extruders, or ram extruders. The binder and the primary ingredient may be fed into the feed hopper of an extruder, heating the materials to melt the binder and form a uniform dispersion or bend with the primary ingredient, extruding the mixture through the die of the extruder to form a thin strand, cooling the extruded strand to solidify the binder and encapsulate the primary ingredient, and cutting, grinding, or milling the solidified strand into particles of the desired size range, which may then be further refined or classified as desired. The encapsulated primary ingredients from hot melt extrusion may be further coated by other means, such as spray drying, to impart a relatively temperature resistant coating on the complex provided by hot melt extrusion.

Electrospinning may be used to apply fine fibers such as nanofibers to cover all or part of the surface of a particle with a suitable material such as a polysaccharide or polymer. Electrospinning as a method for encapsulation may include electrohydrodynamic atomization of the active during the electrospinning of fibers which may be nanofibers, or otherwise combining a primary ingredient with electrospun fibers by coating, entanglement, electrostatic attraction, adhesion, coating, or embedding. Electrospun materials tend to have large surface area and 3D continuous structure that may be manipulated to create broad ranges of filament widths, mesh geometry, fabric density and porosity. Electrospinning approaches may include, by way of example, airblowing-assisted, multi-nozzle, pressurized gyration, needleless, corona, and centrifugal spinning techniques. Melt spinning may be done at lower temperatures than is typical for spray drying or other techniques, including at room temperature or slightly elevated temperatures, and often avoid the temperature ranges that may be harmful for some primary ingredients.

One or more of the above encapsulation methods may be applied sequentially to add multiple layers of coating materials to primary ingredient particles. For example, in a Wurster coater, particles treated with a first encapsulant may be dried and then circulated in the same or a different fluidized bed and treated with a second encapsulant, and this may be repeated as many times as desired to apply multiple layers to particles of primary ingredients. As a further example, a first coating may be applied with one method such as a spray dryer, and a second layer may be applied by a different method such as a fluidized bed, and a third layer may be applied by electrospinning or, for example, by solvent evaporation to apply a material such as shellac from a solution in ethanol or other solvents.

The palatable chewable composition further includes a matrix, in which the encapsulated primary ingredient is dispersed. The matrix includes a sweetener, a hydrocolloid, and a solvent.

In some embodiments, the matrix may further include: one or more emulsifiers (e.g. lecithin, monoglycerides and diglycerides, oleates, Acacia gum, saponins, sucrose fatty acid esters, polysorbates, sodium lauryl sulfate, potassium laurate, bentonite, magnesium hydroxide, or glycerin fatty acid ester), one or more surfactants (e.g. wetting agents (e.g. sorbitan monolaurate), one or more antifoaming agents (e.g. cetostearyl alcohol, lemon oil or other citrus oils, castor oil and other oils, sorbitan trioleate), one or more detergents (e.g. sucrose stearate)), one or more solubilizing agents (e.g. polysorbates such as polysorbate 80, propylene glycol, povidone or polyvinylpyrrolidone, cyclodextrins, glycerin or glycerol, polyethylene glycols or PEGs, and some alcohols such as ethanol, polyethylene glycol 400 monostearate), one or more buffering agents (e.g., phosphates, citrates, lactates), one or more stabilizing agents such as antioxidants (e.g., ascorbic acid, N-acetyl cysteine, propionic acid, sodium bisulfite, sodium sulfite), one or more chelating agents (e.g., tartaric acid, phytic acid, EDTA, malic acid, ascorbic acid, citric acid, amino acids, fumaric acid, sodium edetate), one or more glidants (e.g., silicon dioxide, stearic acid, magnesium stearate, calcium stearate, starches (e.g. tapioca, pea or corn starch), one or more colorants, one or more sweeteners, one or more processing aids (e.g. vegetable oils and other lubricating agents, including stearic acid, glyceryl behenate, calcium stearate, magnesium stearate, zinc stearate, mineral and vegetable oils, benzoic acid, polyethylene glycol), one or more thickeners and suspending agents (e.g. alginates, xanthan gum, guar gum, carboxymethylcellulose, methylcellulose, ethyl cellulose), one or more synthetic polymers (e.g. carbomers, polyvinyl pyrrolidone), one or more clays (e.g. bentonite, montmorillonite, magnesium aluminum silicate, hectorite), one or more preservatives (e.g., benzoic acid, benzalkonium chloride, benzethonium chloride, sodium benzoate, potassium sorbate and sorbic acid), one or more opaquing agents (e.g., titanium dioxide), one or more perfuming agents (e.g., essential oils), one or more glazing agents (e.g., cocoa wax, vegetable oil, beeswax, carnauba wax), one or more flavoring agents, or any combination thereof.

The composition of the manufactured palatable chewable composition matrix will generally differ from the recipe used to create the liquid matrix solution due to changes during manufacturing, particularly a loss of water or other solvent as significant amounts of water may be removed during heating. For example, a gelatin-based matrix may be prepared by heating an aqueous solution of gelatin and glycerin for a time sufficient to remove from about 10% to about 80% of the initial moisture content of the aqueous solution.

The matrix may be present in the palatable chewable composition in an amount from about 60% to about 99% by weight of the composition. The matrix may be from about 60% to about 65%, about 60% to about 70%, about 60% to about 75%, about 60% to about 80%, about 60% to about 85%, about 60% to about 90%, about 60% to about 92%, about 60% to about 94%, about 60% to about 96%, about 65% to about 70%, about 65% to about 75%, about 65% to about 80%, about 65% to about 85%, about 65% to about 90%, about 65% to about 92%, about 65% to about 94%, about 65% to about 96%, about 70% to about 75%, about 70% to about 80%, about 70% to about 85%, about 70% to about 90%, about 70% to about 92%, about 70% to about 94%, about 70% to about 96%, about 75% to about 80%, about 75% to about 85%, about 75% to about 90%, about 75% to about 92%, about 75% to about 94%, about 75% to about 96%, about 80% to about 85%, about 80% to about 90%, about 80% to about 92%, about 80% to about 94%, about 80% to about 96%, about 85% to about 90%, about 85% to about 92%, about 85% to about 94%, about 85% to about 96%, about 90% to about 92%, about 90% to about 94%, about 90% to about 96%, about 92% to about 94%, about 92% to about 96%, about 94% to about 96%, about 94% to about 96%, about 96% to about 98%, about 94% to about 96%, about 96% to about 98%, or about 98% to about 99% by weight of the palatable chewable composition. For example, the matrix may be present in the palatable chewable composition in an amount of about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or about 99% by weight of the palatable chewable composition.

In some embodiments, the matrix may be from about 60% to about 61%, about 60% to about 62%, about 60% to about 63%, about 60% to about 64%, about 60% to about 65%, about 60% to about 66%, about 60% to about 67%, about 60% to about 68%, about 60% to about 69%, about 60% to about 70%, about 60% to about 71%, about 60% to about 72%, about 60% to about 73%, about 60% to about 74%, about 60% to about 75%, about 60% to about 76%, about 60% to about 77%, about 60% to about 78%, about 60% to about 79%, about 60% to about 80%, about 60% to about 81%, about 60% to about 82%, about 60% to about 83%, about 60% to about 84%, about 60% to about 85%, about 60% to about 86%, about 60% to about 87%, about 60% to about 88%, about 60% to about 89%, about 60% to about 90%, about 60% to about 91%, about 60% to about 92%, about 60% to about 93%, about 60% to about 94%, about 60% to about 95%, about 60% to about 96%, about 61% to about 62%, about 61% to about 63%, about 61% to about 64%, about 61% to about 65%, about 61% to about 66%, about 61% to about 67%, about 61% to about 68%, about 61% to about 69%, about 61% to about 70%, about 61% to about 71%, about 61% to about 72%, about 61% to about 73%, about 61% to about 74%, about 61% to about 75%, about 61% to about 76%, about 61% to about 77%, about 61% to about 78%, about 61% to about 79%, about 61% to about 80%, about 61% to about 81%, about 61% to about 82%, about 61% to about 83%, about 61% to about 84%, about 61% to about 85%, about 61% to about 86%, about 61% to about 87%, about 61% to about 88%, about 61% to about 89%, about 61% to about 90%, about 61% to about 91%, about 61% to about 92%, about 61% to about 93%, about 61% to about 94%, about 61% to about 95%, about 61% to about 96%, about 62% to about 63%, about 62% to about 64%, about 62% to about 65%, about 62% to about 66%, about 62% to about 67%, about 62% to about 68%, about 62% to about 69%, about 62% to about 70%, about 62% to about 71%, about 62% to about 72%, about 62% to about 73%, about 62% to about 74%, about 62% to about 75%, about 62% to about 76%, about 62% to about 77%, about 62% to about 78%, about 62% to about 79%, about 62% to about 80%, about 62% to about 81%, about 62% to about 82%, about 62% to about 83%, about 62% to about 84%, about 62% to about 85%, about 62% to about 86%, about 62% to about 87%, about 62% to about 88%, about 62% to about 89%, about 62% to about 90%, about 62% to about 91%, about 62% to about 92%, about 62% to about 93%, about 62% to about 94%, 62% to about 95%, and 62% to about 96%, about 63% to about 64%, 63% to about 65%, about 63% to about 66%, about 63% to about 67%, about 63% to about 68%, about 63% to about 69%, about 63% to about 70%, about 63% to about 71%, about 63% to about 72%, about 63% to about 73%, about 63% to about 74%, about 63% to about 75%, about 63% to about 76%, about 63% to about 77%, about 63% to about 78%, about 63% to about 79%, about 63% to about 80%, about 63% to about 81%, about 63% to about 82%, about 63% to about 83%, about 63% to about 84%, about 63% to about 85%, about 63% to about 86%, about 63% to about 87%, about 63% to about 88%, about 63% to about 89%, about 63% to about 90%, about 63% to about 91%, about 63% to about 92%, about 63% to about 93%, about 63% to about 94%, about 63% to about 95%, about 63% to about 96%, about 64% to about 65%, about 64% to about 66%, about 64% to about 67%, about 64% to about 68%, about 64% to about 69%, about 64% to about 70%, about 64% to about 71%, about 64% to about 72%, about 64% to about 73%, about 64% to about 74%, about 64% to about 75%, about 64% to about 76%, about 64% to about 77%, about 64% to about 78%, about 64% to about 79%, about 64% to about 80%, about 64% to about 81%, about 64% to about 82%, about 64% to about 83%, about 64% to about 84%, about 64% to about 85%, about 64% to about 86%, about 64% to about 87%, about 64% to about 88%, about 64% to about 89%, about 64% to about 90%, about 64% to about 91%, about 64% to about 92%, about 64% to about 93%, about 64% to about 94%, about 64% to about 95%, about 64% to about 96%, about 65% to about 66%, about 65% to about 67%, about 65% to about 68%, about 65% to about 69%, about 65% to about 70%, about 65% to about 71%, about 65% to about 72%, about 65% to about 73%, about 65% to about 74%, about 65% to about 75%, about 65% to about 76%, about 65% to about 77%, about 65% to about 78%, about 65% to about 79%, about 65% to about 80%, about 65% to about 81%, about 65% to about 82%, about 65% to about 83%, about 65% to about 84%, about 65% to about 85%, about 65% to about 86%, about 65% to about 87%, about 65% to about 88%, about 65% to about 89%, about 65% to about 90%, about 65% to about 91%, about 65% to about 92%, about 65% to about 93%, about 65% to about 94%, about 65% to about 95%, about 65% to about 96%, about 66% to about 67%, about 66% to about 68%, about 66% to about 69%, about 66% to about 70%, about 66% to about 71%, about 66% to about 72%, about 66% to about 73%, about 66% to about 74%, about 66% to about 75%, about 66% to about 76%, about 66% to about 77%, about 66% to about 78%, about 66% to about 79%, about 66% to about 80%, about 66% to about 81%, 66% to about 82%, about 66% to about 83%, about 66% to about 84%, about 66% to about 85%, about 66% to about 86%, about 66% to about 87%, about 66% to about 88%, about 66% to about 89%, about 66% to about 90%, about 66% to about 91%, about 66% to about 92%, about 66% to about 93%, about 66% to about 94%, about 66% to about 95%, about 66% to about 96%, about 67% to about 68%, about 67% to about 69%, about 67% to about 70%, about 67% to about 71%, about 67% to about 72%, about 67% to about 73%, about 67% to about 74%, about 67% to about 75%, about 67% to about 76%, about 67% to about 77%, 67% to about 78%, about 67% to about 79%, about 67% to about 80%, about 67% to about 81%, 67% to about 82%, about 67% to about 83%, about 67% to about 84%, about 67% to about 85%, about 67% to about 86%, about 67% to about 87%, about 67% to about 88%, about 67% to about 89%, about 67% to about 90%, about 67% to about 91%, about 67% to about 92%, about 67% to about 93%, about 67% to about 94%, about 67% to about 95%, about 67% to about 96%, about 68% to about 69%, about 68% to about 70%, about 68% to about 71%, about 68% to about 72%, about 68% to about 73%, about 68% to about 74%, about 68% to about 75%, about 68% to about 76%, about 68% to about 77%, about 68% to about 78%, about 68% to about 79%, about 68% to about 80%, about 68% to about 81%, about 68% to about 82%, about 68% to about 83%, about 68% to about 84%, about 68% to about 85%, about 68% to about 86%, about 68% to about 87%, about 68% to about 88%, about 68% to about 89%, about 68% to about 90%, about 68% to about 91%, about 68% to about 92%, about 68% to about 93%, about 68% to about 94%, about 68% to about 95%, about 68% to about 96%, about 69% to about 70%, about 69% to about 71%, about 69% to about 72%, about 69% to about 73%, about 69% to about 74%, about 69% to about 75%, about 69% to about 76%, about 69% to about 77%, about 69% to about 78%, about 69% to about 79%, about 69% to about 80%, about 69% to about 81%, about 69% to about 82%, about 69% to about 83%, about 69% to about 84%, about 69% to about 85%, about 69% to about 86%, about 69% to about 87%, about 69% to about 88%, about 69% to about 89%, about 69% to about 90%, about 69% to about 91%, about 69% to about 92%, about 69% to about 93%, about 69% to about 94%, about 69% to about 95%, about 69% to about 96%, about 70% to about 71%, about 70% to about 72%, about 70% to about 73%, about 70% to about 74%, about 70% to about 75%, about 70% to about 76%, about 70% to about 77%, about 70% to about 78%, about 70% to about 79%, about 70% to about 80%, about 70% to about 81%, about 70% to about 82%, about 70% to about 83%, about 70% to about 84%, about 70% to about 85%, about 70% to about 86%, about 70% to about 87%, about 70% to about 88%, about 70% to about 89%, about 70% to about 90%, about 70% to about 91%, about 70% to about 92%, about 70% to about 93%, about 70% to about 94%, about 70% to about 95%, or about 70% to about 96% by weight of the palatable chewable composition.

The matrix includes a sweetener. A wide variety of sweeteners may be used in the palatable chewable composition of the present disclosure, such as sugar, sucrose, glucose, maltose, galactose, psicose, isomaltulose, tagatose, trehalose, raffinose, fructose, sorbose, sugar alcohol, honey, fruit products (e.g., fruit extracts, fruit slurries, dried fruit, or fruit juice), agave, maple syrup, Stevia, allulose, monk fruit extract, xylitol, corn syrup, corn syrup solids, high fructose corn syrup, dextrose, saccharin, maltodextrin, aspartame, potassium acesulfame (acesulfame-K or Ace-K), sucralose, neotame, advantame, neohesperidin dihydrochalcone, or any combination thereof. In some embodiments, the sweetener may be glucose, sucralose, sucrose, corn syrup, corn syrup solids, or any combination thereof. Tartness and a desired pH may be provided by including citric acid or other ingredients described above.

In some embodiments, the palatable chewable composition may include from about 30% to about 80% sweetener by weight of the palatable chewable composition. The amount of sweetener may be from about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, about 35% to about 40%, about 35% to about 45%, about 35% to about 50%, about 35% to about 55%, about 35% to about 60%, about 35% to about 65%, about 35% to about 70%, about 35% to about 75%, about 35% to about 80%, about 40% to about 45%, about 40% to about 50%, about 40% to about 55%, about 40% to about 60%, about 40% to about 65%, about 40% to about 70%, about 40% to about 75%, about 40% to about 80%, about 45% to about 50%, about 45% to about 55%, about 45% to about 60%, about 45% to about 65%, about 45% to about 70%, about 45% to about 75%, about 45% to about 80%, about 50% to about 55%, about 50% to about 60%, about 50% to about 65%, about 50% to about 70%, about 50% to about 75%, about 50% to about 80%, 55% to about 60%, about 55% to about 65%, about 55% to about 70%, about 55% to about 75%, about 55% to about 80%, about 60% to about 65%, about 60% to about 70%, about 60% to about 75%, about 60% to about 80%, about 65% to about 70%, about 65% to about 75%, about 65% to about 80%, about 70% to about 75%, about 70% to about 80%, or from about 75% to about 80% by weight. For example, the weight percent of sweetener may range from about 35% to about 55%, about 40% to about 50%, from about 31% to about 59%, from about 32% to about 58%, from about 33% to about 57%, from about 34% to about 56%, from about 35% to about 55%, from about 36% to about 54%, from about 37% to about 53%, from about 38% to about 52%, from about 39% to about 51%, from about 40% to about 50%, from about 41% to about 48%, from about 42% to about 47%, from about 43% to about 46%, or from about 44% to about 45% by weight of the palatable chewable composition. As another example, the palatable chewable composition may include about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or about 80% sweetener by weight of the palatable chewable composition.

The matrix further includes a hydrocolloid. In certain embodiments, a hydrocolloid may refer to a water-soluble polymeric material capable of forming a viscous dispersion and/or gel upon hydration. Hydrocolloids may be obtained from a variety of sources, including plant-derived materials (e.g., starches, pectins, guar gum, locust bean gum, tamarind seed gum, konjac glucomannan), seaweed-derived materials (e.g., agar, carrageenan, alginates), microbial fermentation products (e.g., xanthan gum, gellan gum, pullulan), and animal-derived materials (e.g., gelatin, chitosan). In additional embodiments, hydrocolloids may comprise semi-synthetic or chemically modified derivatives, including but not limited to carboxymethyl cellulose, methylcellulose, hydroxypropylmethylcellulose, and microcrystalline cellulose. Such hydrocolloids are useful for modifying rheological properties, stabilizing dispersions or emulsions, forming films or gels, and providing encapsulation or controlled-release functionality in pharmaceutical, food, cosmetic, and biomedical applications.

In some embodiments, the hydrocolloid may include a gelling agent. The gelling agent may contribute to the chewable texture and structural integrity of the composition. Examples of gelling agents include, but are not limited to pectin, gelatin, alginates, agar, carrageenan, tragacanth, xanthan gum, methylcellulose, hydroxypropyl methylcellulose (HPMC), carboxymethylcellulose (CMC), carbomers (Carbopol®), poloxamers (Pluronics®), or polyvinyl alcohol (PVA).

In some embodiments, the hydrocolloid may comprise carrageenan, gelatin, pectin, agar, starch, locust bean gum, konjac, gum Arabic, sago, or any combination thereof.

The hydrocolloid may be present in the palatable chewable composition in an amount from about 0.5% to about 15% by weight of the composition. The amount of hydrocolloid may be from about 0.5% to about 1%, about 0.5% to about 2%, about 0.5% to about 3%, about 0.5% to about 5%, about 0.5% to about 7%, about 0.5% to about 10%, about 0.5% to about 12%, about 0.5% to about 14%, about 1% to about 3%, about 1% to about 5%, about 1% to about 7%, about 1% to about 10%, about 1% to about 12%, about 1% to about 15%, about 2% to about 5%, about 2% to about 7%, about 2% to about 10%, about 2% to about 12%, about 2% to about 15%, about 3% to about 5%, about 3% to about 7%, about 3% to about 10%, about 3% to about 12%, about 3% to about 15%, about 5% to about 7%, about 5% to about 10%, about 5% to about 12%, about 5% to about 15%, about 7% to about 10%, about 7% to about 12%, about 7% to about 15%, about 10% to about 12%, about 10% to about 15%, or about 12% to about 15% by weight of the palatable chewable composition. As another example, the hydrocolloid may be present in the palatable chewable composition in an amount of about 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, or about 14% by weight of the palatable chewable composition.

In some embodiments, the hydrocolloid is carrageenan. The carrageenan may be present in an amount from about 0.5% to about 4% by weight of the palatable chewable composition. The amount of carrageenan may be from about 0.5% to about 1%, about 0.5% to about 1.5%, about 0.5% to about 2%, about 0.5% to about 2.5%, about 0.5% to about 3%, about 0.5% to about 3.5%, about 0.5% to about 4%, about 1% to about 1.5%, about 1% to about 2%, about 1% to about 2.5%, about 1% to about 3%, about 1% to about 3.5%, about 1% to about 4%, about 1.5% to about 2%, about 1.5% to about 2.5%, about 1.5% to about 3%, about 1.5% to about 3.5%, about 1.5% to about 4%, about 2% to about 2.5%, about 2% to about 3%, about 2% to about 3.5%, about 2% to about 4%, about 2.5% to about 3%, about 2.5% to about 3.5%, about 2.5% to about 4%, about 3% to about 3.5%, about 3% to about 4%, or about 3.5% to about 4% by weight of the palatable chewable composition. As another example, the amount of carrageenan may be about 0.5%, 0.6%, 0.7%, 0.8%, 0.9%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, or about 4% by weight of the palatable chewable composition.

The matrix further includes a solvent. The solvent may be water, glycerin, alcohol, glycol, medium chain triglycerides (MCT), propylene glycol (PG), or other pharmaceutically acceptable or food-grade solvent or any combination thereof. In some embodiments, the solvent may be water.

The solvent may be present in the palatable chewable composition in an amount from about 5% to about 30% by weight. The solvent may range from about 5% to about 6%, about 6% to about 7%, about 7% to about 8%, about 9% to about 10%, about 10% to about 12%, 10% to about 15%, about 10% to about 18%, about 10% to about 20%, about 10% to about 22%, about 10% to about 25%, about 10% to about 28%, about 10% to about 30%, about 12% to about 15%, about 12% to about 18%, about 12% to about 20%, about 12% to about 22%, about 12% to about 25%, about 12% to about 28%, about 12% to about 30%, about 15% to about 18%, about 15% to about 20%, about 15% to about 22%, about 15% to about 25%, about 15% to about 28%, about 15% to about 30%, about 18% to about 20%, about 18% to about 22%, about 18% to about 25%, about 18% to about 28%, about 18% to about 30%, about 20% to about 22%, about 20% to about 25%, about 20% to about 28%, about 20% to about 30%, about 22% to about 25%, about 22% to about 28%, about 22% to about 30%, about 25% to about 28%, about 25% to about 30%, or about 28% to about 30% by weight of the palatable chewable composition. As another example, the solvent may be present in the palatable chewable composition in an amount of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or about 30% by weight of the palatable chewable composition.

In some embodiments, the amount of the solvent may be from about 5% to about 29%, about 7% to about 29%, about 9% to about 29%, about 11% to about 29%, about 12% to about 28%, about 13% to about 27%, about 14% to about 26%, about 15% to about 25%, about 16% to about 24%, about 17% to about 23%, about 18% to about 22%, about 19% to about 21%, or about 19.5% to about 20% by weight of the palatable chewable composition.

The palatable chewable composition may further include a flavoring agent. The flavoring agent may comprise a synthetic flavoring agent. In some embodiments, the synthetic flavoring agent may include cherry, strawberry acai, strawberry peach, fruit punch, or orange.

In some embodiments, the palatable chewable composition product may include about 0.01% to about 5% flavoring agent by weight of the palatable chewable composition. The flavoring agent may range from about 0.01% to about 4%, from about 0.01% to about 3%, from about 0.01% to about 2%, from about 0.01% to about 1%, or from about 0.01% to about 0.5% by weight of the palatable chewable composition. In some embodiments, the flavoring agent may range from about 0.01% to about 5%, from about 0.05% to about 5%, from about 0.1% to about 5%, from about 0.25% to about 5%, from about 0.5% to about 5%, or from about 1% to about 5% by weight of the palatable chewable composition. The flavoring agent may be present in an amount from about 0.05% to about 2%, from about 0.1% to about 1%, or from about 0.25% to about 0.75% by weight of the palatable chewable composition.

In some embodiments, the humectant may be glycerin.

In some embodiments, glycerin may be used as a humectant, calcium carbonate as a filler, coconut oil as a lipid, lecithin as an emulsifier, and citric acid, malic acid, or sodium citrate as pH modifiers. Color additives may be artificial or natural food colors, depending on formulation preferences.

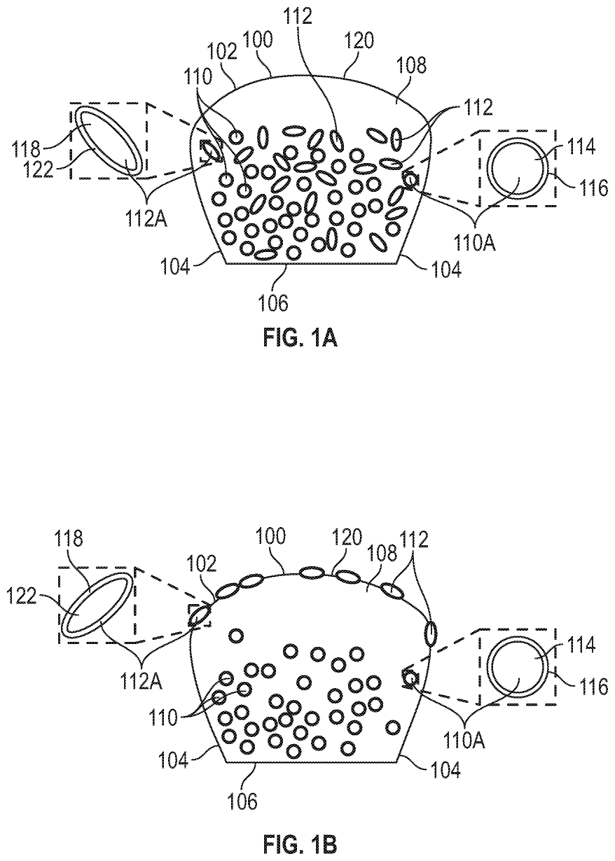

FIG. 1A depicts a cross-section of an embodiment of a palatable chewable composition 100 having an outer surface 102 with a top surface 120, side surfaces 104, and a bottom surface 106. Embedded in the matrix 108 is a first encapsulated active 110 depicted as a plurality of similar coated spheres, and a second encapsulated active 112 depicted as a plurality of coated ellipsoids. One or both of the first and second encapsulated primary ingredients 110, 112 may have an unpleasant taste and/or unpleasant odor that should be masked. The first encapsulated active 110A is shown with an enlarged view, revealing the first active encapsulated core 114 and its coating layer 116, which may be a multilayer or multicomponent coating including polymers, lipids, nanofibers, shellac, and the like. Likewise, a characteristic second encapsulated active 112A is shown with an enlarged view, revealing the second active encapsulated core 118 and its coating layer 122, which also may be a multilayer or multicomponent coating. The coating layer 116 of the first encapsulated active 110A may not be the same as the coating layer 122 of the second encapsulated active, but may differ in terms of material, number of layers, thickness of the layer, porosity of the layers. For example, first encapsulated active 110 may be coated in a manner designed to release it rapidly in the mouth and stomach, while the second encapsulated active 112 may be coated in a manner designed to release it slowly over two or more hours in the small and large intestines.

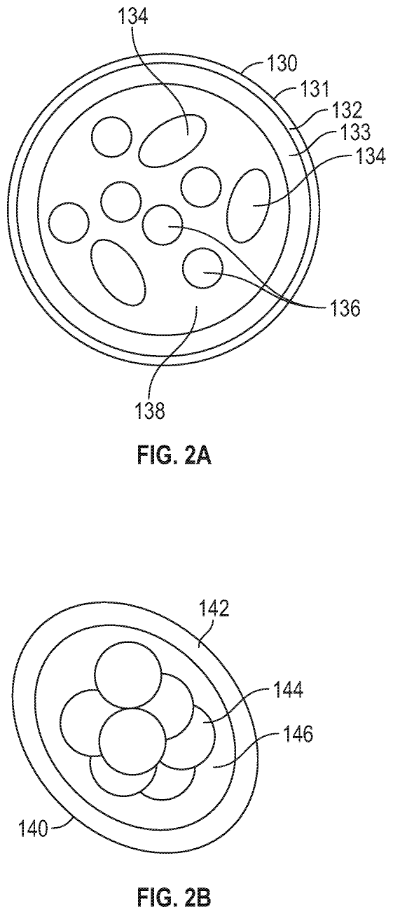

FIG. 1B depicts a cross-section of an embodiment of a palatable chewable composition 100 similar to that of FIG. 1A, but here the second encapsulated active 112, again depicted as a plurality of coated ellipsoids, are largely on the outer surface 102 of the palatable chewable composition 100 and particularly on the top surface 120. A characteristic second encapsulated active 112A is shown with an enlarged view, revealing the second active encapsulated core 118 and its coating layer 122. Such a palatable chewable composition 100 may be useful then there is a risk of interaction between the first encapsulated active 110 and the second encapsulated active 112, such that the two primary ingredients 110, 112 are now relatively more segregated than was the case in FIG. 2A. Other reasons for placing one active on the outer surface 102 and the other inside the matrix 108 may include a desire to have the first encapsulated active 110 be released quickly beginning in the mouth, or a desire to have it influence the flavor that will be perceived during chewing of the palatable chewable composition 100 and possible release of some of the second encapsulated active 112. In some cases, the second encapsulated active 112 may be particularly suitable for combination with a flavoring, a sugar or sugar alcohol such as xylitol which may provide a cooling effect, or acid such as citric acid or malic acid that may be beneficial in terms of the taste experience if provided with the second encapsulated active 112 on the outer surface 102 of the palatable chewable composition 100.

FIGS. 2A to 2B depict different encapsulated materials. FIG. 2A shows an encapsulated complex 130 having an external dual-layer coating 131 (though it may also have only one layer or more than two layers) having a first layer 133 and a concentric second layer 132, which collectively encapsulate an internal medium 138 (or this may be termed a “secondary matrix” when the encapsulated complex 130 is itself embedded within another matrix in a palatable chewable composition, not shown). The internal medium 138 itself may have multiple materials disposed as concentric layers, bi-continuous mixtures, island-and-sea mixtures. Within the internal medium 138 is embedded a first primary ingredient 134 depicted as ellipsoids and a second primary ingredient 136 depicted as spheres. The internal medium 138 may be a liquid, a syrup, a slurry, a gelled material or partially gelled material similar to or different than the matrix (not shown) of a palatable chewable composition (not shown), or could comprise lipids such as waxes or oils, hydrophobic barrier materials, chewable solids, edible oxygen scavengers, antioxidants such as N-acetyl cysteine or ascorbic acid, preservatives, complexing agents, which may be adapted to assist in the flavor/aroma masking of, or to enhance the stability of, the first and second primary ingredients 134, 136. In some aspects, second layer 132 or both layers 132, 133 of the dual-layer coating 131 of the encapsulated complex 130 may be relatively insensitive to at least one of elevated temperature, moisture level, a pH range of concern (e.g., an acidic pH of matrix of a palatable chewable composition that might otherwise be harmful to the stability of one or more contents of the encapsulated complex 130). Thus, in one aspect, the second layer 132 and/or first layer 133 of the dual-layer coating 131 may be shellac or other relatively rigid polymers or other materials capable of resisting at least one of (1) dissolution in an aqueous matrix solution (not shown) during manufacturing; (2) shear during the mixing process with the aqueous matrix solution) during manufacturing; (3) the pH of the aqueous matrix solution which may be acidic such as having a pH from 3 to 5.5; (4) migration of significant amounts of moisture from the matrix into the first primary ingredient 134 or second primary ingredient 136 during manufacturing or storage of the palatable chewable composition (not shown); (5) oxygen transport into the first primary ingredient 134 or second primary ingredient 136; and (6) elevated temperature in the manufacturing process (e.g., holding a low-melting point internal medium 138 in place when it temporarily liquifies when in contact with a hot aqueous solution during manufacturing), in addition to also providing a benefit with respect to flavor or aroma masking. The latter benefit may be provided by preventing the entry of moisture into the internal medium, reducing diffusion of a primary ingredient or flavor or aroma component thereof from within the internal medium 138 through the dual-layer coating 131, preventing reaction of either the first primary ingredient 134 or the second primary ingredient 136 with a component in the external environment (not shown) such as the matrix of a palatable chewable composition or the aqueous matrix solution during manufacturing, such as prevent premature contact with an acidic material whose low pH might result in release of odorants or bitter materials. As a result, the dual-layer coating 131, and/or the internal medium 138 can help protect the stability of the primary ingredient while also maintaining efficacy of masking of flavors or odors.

In one aspect, the dual-layer coating 131 may have a water-resistant layer that may include a polymer coating such as polyethylene or polymethacrylates or other acrylate polymers, which may be prepared in an aqueous dispersion that is dried during or after application on a spray dryer or other encapsulation process, resulting in film formation during drying that can provide a water impervious or water resistant layer, whose properties may be adjusted by combination with other agents such as salts and/or fillers during coating. Both or either of the first primary ingredient 134 and second primary ingredient 136 may also be individually encapsulated (not shown) with suitable flavor or odor masking materials or water-resistant materials.

FIG. 2B shows another encapsulated complex 140 having a coating layer 142, agglomerated particles 144 that may comprise one or more primary ingredients or other agents, and an internal medium 146 which may be a liquid such as an edible viscous liquid (e.g., having a viscosity at room temperature of at least 1000 cps or 5000 cps or 10,000 cps such as from 5,000 cps to 80,000 cps), a slurry, a fatty material having one or more lipids, a gelled material similar or dissimilar to the matrix (not shown) of a palatable chewable composition product (not shown). The coating layer 142, in combination with the internal medium 148, or the internal medium 148 itself may serve to mask the odor or flavor of the agglomerated particles 144 and may also enhance the stability of the agglomerated particles 144. For example, the agglomerated particles 144 may be sensitive to shear, which could otherwise cause them to disperse during manufacturing and affect the flavor of the palatable chewable composition product or cause them to be prematurely released, dissolve in the matrix solution or be damaged by interaction with one or more agents in the matrix or the matrix solution (not shown). Protecting the agglomerated particles 144 with both a secondary matrix 146 and a coating layer 142 increases resistance to destabilizing challenges such as shear, oxidation, moisture, and/or pH.

The multi-layer coating may generally provide enhanced stability that may be jeopardized during the manufacturing process of the palatable chewable composition. The multi-layer coating may additionally provide enhanced stability to the primary ingredient from the properties of the solution or solutions used to produce the matrix or by long-term contact with the matrix of the palatable chewable composition.

The palatable chewable composition matrix may be typically made from a heated aqueous solution having sweeteners, hydrocolloids, flavorings, pH control agents, and the like. Moisture from the solution or the gelled matrix may be harmful to many primary ingredients. pH range and other aspects of the materials in contact with encapsulated primary ingredients can also be harmful to many primary ingredients. Further, the elevated temperature of the aqueous solution when the primary ingredients are blended in may also be harmful to enteric coatings on the primary ingredients or otherwise damage the primary ingredients. Furthermore, high shear during blending may damage the active or its coating. But with the methods, compositions, and processes described herein, palatable chewable compositions may be provided in which the primary ingredients and their coatings are protected from the manufacturing process and achieve higher stability than is possible with conventional approaches, resulting in palatable chewable compositions that are more efficacious in delivering one or more primary ingredients as intended to provide benefits to the user, while still also having the desired masking benefit relative to one or more unpalatable flavors or malodors.

The coating in the palatable chewable composition helps maintain the encapsulated primary ingredient's pharmaceutical performance characteristics when the primary ingredient is an active pharmaceutical ingredient. The pharmaceutical performance characteristics include efficacy. Shear and oxidation are manufacturing or environmental factors that may degrade the pharmaceutical performance characteristics.

### Method of Making the Palatable Chewable Composition

Gums, jellies, and pastilles constitute a large class of confectionery products which may be manufactured with many interesting variations. They are comparatively low boiled and contain about 20% moisture. The texture of these products, which may be firm or solid, may be obtained by the use of various types of water-binding gelling agents principally gum Arabic, starch, gelatine, agar, and pectin. After a boiled mixture of mixed sugars has been prepared it may be mixed with the gelling agent and then processed into the range of shapes by depositing into starch moulds. Other methods may be used, principally depositing into rubber moulds or pouring onto a slab. After forming into shapes, the confections are dried to their final moisture content and texture by stoving.

Further provided herein are methods of making a palatable chewable composition. The palatable chewable composition may be any of the palatable chewable compositions described hereinabove. The method comprises combining a sweetener, a hydrocolloid, and a solvent to form a homogeneous mixture; conditioning the homogenous mixture temperature to a temperature from about 40° C. to about 100° C.; mixing in an encapsulated primary ingredient to the homogenous mixture; depositing the homogeneous mixture into molds; and cooling the deposited homogenous mixture to form the palatable chewable composition. In some embodiments, the homogeneous mixture does not include a hydrocolloid.

Combining the sweetener, the hydrocolloid (when present), and the solvent to form the homogeneous mixture may be accomplished by combining each ingredient in a vessel while mixing. All ingredients may be added to a vessel before mixing begins, or the ingredients may be added sequentially as mixing is conducted. Mixing apparatuses known in the art may be used to mix the ingredients to form a homogeneous mixture; for example, a stand mixer, a paddle mixer, ribbon blender, planetary mixer, high shear mixer, or any other mixing apparatus known in the art. The mixture may be determined to be homogeneous by a visual inspection. The mixing may be conducted at ambient or elevated temperatures for ease of processing and hydrocolloid hydration.

In some embodiments, one or more additional ingredients may be incorporated into the homogeneous mixture. For example, during the initial combining step, additional ingredients selected from non-encapsulated active pharmaceutical ingredients, color additives, humectants, gelling agents, pH modifiers, and combinations thereof may be included. Alternatively, one or more of these additional ingredients may be added to the cooled mixture after heating, prior to molding. Humectants may include glycerin, sorbitol, propylene glycol, sodium lactate, or honey, which help retain moisture and improve chewability. Gelling agents may include gelatin, pectin, or agar, which contribute to structural integrity. Color additives may be natural or synthetic dyes or pigments, pH modifiers such as citric acid, sodium citrate, phosphates, or ascorbic acid may be used to optimize taste, stability, or solubility of the API. These ingredients may be in solid, liquid, or slurry form.

The encapsulated primary ingredient may be added to the homogeneous mixture slowly or all at once. The homogeneous mixture may be continuously mixed to ensure the mixture maintains homogeneity throughout the course of the mixing and to ensure adequate dispersal of the encapsulated primary ingredient. Mixing may be performed under low shear or moderate shear conditions sufficient to evenly disperse the encapsulated API within the slurry. In certain embodiments, mixing may be performed for 120 minutes or less, for example, from about 15 seconds to about 90 minutes, depending on viscosity and batch size.

Before or after the encapsulated primary ingredient is added, the temperature of the homogeneous mixture may be conditioned. Adjusting or conditioning the temperature may include heating or cooling the homogeneous mixture. The temperature may be lower than a decomposition temperature of the primary ingredient; i.e., the temperature at which the primary ingredient begins to chemically decompose or the encapsulant begins to decompose, thereby allowing water and other materials to interact with the primary ingredient and to reduce the taste-masking ability of the encapsulant. The decomposition temperatures of the encapsulated primary ingredient may be determined by methods including Differential Scanning calorimetry (DSC) or Hot Stage Microscopy. Further, it will be understood by the skilled artisan that an encapsulated primary ingredient may be conditioned to or near the decomposition temperature of the primary ingredient for short periods of time to avoid significant decomposition of the primary ingredient (i.e., about 20% decomposition or greater) to maintain a desired concentration of the primary ingredient in the final palatable chewable composition.

In some embodiments, the homogeneous mixture may be conditioned to a temperature from about 40° C. to about 70° C. In such embodiments, the homogeneous mixture may be held at this temperature for 40 minutes or longer, as the primary ingredient may be less likely to decompose at these temperatures. For example, the homogenous mixture may be conditioned to about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., or about 70° C. In some embodiments, the homogenous mixture may be conditioned to about 40° C. to about 50° C., about 40° C. to about 60° C., about 40° C. to about 70° C., about 50° C. to about 60° C., about 50° C. to about 70° C., about 60° C. to about 70° C., about 41° C. to about 69° C., about 42° C. to about 68° C., about 43° C. to about 67° C., about 44° C. to about 66° C., about 45° C. to about 65° C., about 46° C. to about 66° C., about 47° C. to about 64° C., about 48° C. to about 63° C., about 49° C. to about 63° C., about 50° C. to about 62° C., about 51° C. to about 61° C., about 52° C. to about 60° C., about 53° C. to about 59° C., about 54° C. to about 58° C., or about 55° C. to about 57° C.

The homogeneous mixture may be held at the temperature above for 40 minutes or longer, such as about 1 hour, about 2 hours, about 4 hours, or longer. The heating may be accomplished using apparatuses known in the art. For example, a jacketed vessel may be used to heat the homogeneous mixture.

Next, the homogeneous mixture may be deposited into molds that conform to a desired shape and size of the palatable chewable composition. The molds may be silicon molds or another food-safe or pharmaceutical-grade material. The homogeneous mixture may be deposited into the molds directly from the vessel in which the homogeneous mixture was formed, or aliquots of the homogeneous mixture may be removed from the vessel sequentially and deposited into separate molds.

Prior to depositing the homogeneous mixture into molds, coloring and flavoring agents may be added and combined in the homogeneous mixture.

After depositing the mixture into molds, the mixture may be cooled to ambient temperature to allow the palatable chewable composition to form into a solid form. The cooling may take place simply by allowing the deposited mixture to cool in ambient conditions, or cooling apparatuses that control temperature and humidity may be used to reduce the temperature of the surroundings and thereby expedite cooling of the mixture and achieve desired texture profiles. In some embodiments, the cooling may be accomplished in a multi-level cooler.

In another embodiment, the method comprises combining a sweetener, a hydrocolloid, and a solvent to form a homogeneous mixture; conditioning the homogeneous mixture to a temperature of up to about 100° C.; adding an encapsulated primary ingredient to the homogeneous mixture while mixing; depositing the homogeneous mixture into molds within 40 minutes from the time homogeneous mixture is conditioned; and cooling or drying the deposited homogenous mixture to form the palatable chewable composition.

The steps of the method may proceed as described in the embodiments above; however, the homogeneous mixture may only be held at the elevated temperatures for no more than 40 minutes to avoid decomposition of the encapsulated active ingredient. The homogenous mixture may be conditioned to about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., or about 100° C. In some embodiments, the homogenous mixture may be conditioned to about 70° C. to about 100° C., about 70° C. to about 95° C., such as about 70° C. to about 75° C., about 70° C. to about 80° C., about 70° C. to about 85° C., about 70° C. to about 90° C., about 70° C. to about 95° C., about 75° C. to about 80° C., about 75° C. to about 85° C., about 75° C. to about 90° C., about 75° C. to about 95° C., about 75° C. to about 100° C., about 80° C. to about 85° C., about 80° C. to about 90° C., about 80° C. to about 95° C., about 85° C. to about 90° C., about 85° C. to about 95° C., about 90° C. to about 95° C., about 71° C. to about 94° C., about 72° C. to about 93° C., about 73° C. to about 92° C., about 74° C. to about 91° C., about 75° C. to about 90° C., about 76° C. to about 89° C., about 77° C. to about 88° C., about 78° C. to about 87° C., about 79° C. to about 86° C., about 80° C. to about 85° C., about 81° C. to about 84° C., or about 82° C. to about 83° C. In some embodiments, the homogenous mixture may be conditioned to about 95° C.

The homogeneous mixture may be held at the temperature above for 40 minutes or less, such as about 30 minutes, about 20 minutes, about 15 minutes, about 10 minutes, about 5 minutes, about 1 minute, or less.

In some embodiments, the deposited mixture may additionally be dried. The drying may be performed in curing rooms with air circulation, which causes additional evaporation of the solvent.

In some embodiments, the cooled compositions may be sanded with sugar in a coating drum. This final step is optional and may be completed to provide a sweet taste to the compositions.

In some embodiments, the method of making a chewable composition for masking the taste of a primary ingredient comprises combining a sweetener and a solvent to form a homogeneous mixture, adding an encapsulated primary ingredient to the homogeneous mixture while mixing, the primary ingredient selected from the group consisting of an active pharmaceutical ingredient, dietary supplement, food additive, and any combination thereof, conditioning the homogeneous mixture to a temperature from about 40° C. to about 95° C.; and forming the homogeneous mixture into shapes to form the chewable composition.

In some embodiments, the palatable chewable composition may be formed by extruding the composition through a die to produce an elongated strand, rope, or ribbon. The extruded composition may then be cut into individual pieces of a desired length or size to form chewable compositions having a predetermined shape. The cross-sectional shape of the extruded composition may be defined by the die geometry and may include circular, oval, rectangular, square, star-shaped, or irregular profiles. The extrusion and cutting process may be performed while the composition is in a pliable or semi-solid state and may be followed by cooling or setting to retain the formed shape.

In some embodiments, the palatable chewable composition may be deposited, spread, or rolled into one or more sheets having a substantially uniform thickness. The sheet may then be cut, stamped, punched, or otherwise subdivided into individual pieces having a desired shape and size. Cutting may be performed using mechanical cutters, dies, blades, or stamping tools.

The thickness of the sheet and the dimensions of the cut shapes may be selected to control the weight, dose, and mouthfeel of the chewable composition.

In some embodiments, the palatable chewable composition may be deposited into molds to form chewable compositions having a predetermined shape. The molds may include cavities corresponding to the desired final shape of the chewable composition. After deposition, the composition may be cooled, conditioned, or otherwise set within the mold before removal.

Molded shapes may include, without limitation, tablets, gummies, lozenges, or novelty shapes, and may be uniform or varied within a single batch.

The extrusion, sheet-cutting, and molding techniques described herein may be used interchangeably or in combination and may be applied to any of the palatable chewable compositions described in this disclosure, including those comprising encapsulated active ingredients. The selected forming technique does not alter the functional properties of the chewable composition but provides flexibility in manufacturing and product design.

The resulting chewable composition may exhibit desirable organoleptic properties, mechanical strength, and controlled release of the primary ingredient. In certain embodiments, the chewable composition is prepared as a gummy, gel, jelly, or soft chew suitable for pediatric, geriatric, or general patient populations who prefer or require non-tablet compositions.

After the palatable chewable composition has formed, the palatable chewable composition may be coated in a tumbler or other device with other agents (oils, waxes, maltodextrin, starch), and may then be steam heated to prepare for a final sugar coating, if desired, and then packaged, sealed, and labeled.

In some aspects, primary ingredients may be added to a palatable chewable composition by methods other than blending the primary ingredients into the homogeneous mixture. Such alternate methods may be useful if the primary ingredient or its encapsulants are particularly temperature-sensitive. For example, encapsulated or coated primary ingredients as well as uncoated primary ingredients may be incorporated into a palatable chewable composition product by blending into a relatively cooler gelled or partially gelled matrix at a temperature well below the peak temperature during the manufacturing process, or by attachment to the surface of a gelled or gelling matrix, by insertion into a cavity in a gelled matrix or into a matrix solution that has been poured into a mold and is undergoing cooling. In one method, a palatable chewable composition shell is molded (this may involve starch molding with a two-part mold, injection molding, building a shell in stages), and then encapsulated primary ingredients are added, either deposited using gravity, air entrainment, or entrainment in a viscous liquid such as a syrup that is adapted to not dissolve the encapsulants of the primary ingredients. In some aspects after the encapsulated primary ingredients are added, additional matrix material is added to seal the encapsulated primary ingredients and complete the shell. In other aspects, a narrow injector inserts a central filling of encapsulated particles without a need for adding further matrix material. Any remaining gap may be sealed by compressing the palatable chewable composition, heating the palatable chewable composition around the gap, or adding sugar or other materials to close the gap.

In another aspect, the manufacturing process begins with compounding according to predetermined recipes, in which the appropriate amount of raw materials are poured into mixing tanks equipped with mixing, heating, and cooling capabilities. As a batch is prepared, the flowable mixture may be tested to ensure that it has suitable quality. Then the palatable chewable composition is formed, which may be done by transferring the flowable mixture to a starch molding machine such as a Mogul. The starch employed helps prevent the palatable chewable compositions from sticking to the palatable chewable composition molds and may help hold the palatable chewable compositions in place during the drying, cooling, and setting processes, and may absorb moisture from the compositions to help give them the desired texture. The palatable chewable compositions are prepared by filling molds in the Mogul device. The molds may have starch applied before receiving the flowable material. After being formed and cooled in the molds, the starch on the surface of the palatable chewable compositions can reduce tackiness and modify the surface feel and mouth feel as the starch absorbs moisture and more fully bonds with the matrix. In one aspect, hot air may be further applied to prepare the surface of the palatable chewable compositions to interact with the starch or other materials added to the surface. The palatable chewable compositions may then be removed (e.g., by inverting trays to dump palatable chewable compositions onto a moving wire or vibrating metal screen that can act as a sieve) to remove excess starch and expose the palatable chewable compositions to various surface treatments (sprayed with vegetable oil, sprinkled with sugars, tartness agents such as tartaric or citric acid, or other solids). They may be stored and cooled (e.g., for 3 or more hours), further decorated as desired, and packaged.

In another aspect, a palatable chewable composition is prepared by mixing water, gelatin and/or pectin and/or carrageenan and/or agar, sweeteners such as sugar and glucose syrup, or, for sucrose-free palatable chewable compositions, polyols and/or polydextrose, then adding colorant, flavorings and aromas, buffers to form a mixture capable of gelling, then at least one encapsulated primary ingredients and other excipients while the mixture at a predetermined blending temperature that may be less than 100° C., such as less than 90° C., less than 85° C., less than 80° C., less than 75° C., or less than 70° C., and adding pH control agents to achieve a predetermined pH, then depositing the blended mixture on molds that may optionally have a particulate base such as starch or other polysaccharides. The filled molds are then dried and removed from the mold.

Other exemplary matrix compositions may include variants of the following: (1) palatable chewable compositions containing pectin; sweetening agents such as sucrose, erythritol, invert syrup and glycerol; a polyvalent metal salt, such as calcium chloride; an edible acid, such as citric acid, and about 15% water by weight of the palatable chewable composition; (2) from 5% to 30% water by weight of the palatable chewable composition, from 5% to 15% gelling agent such as pectin, gelatin, or carrageenan by weight of the palatable chewable composition, 10% to 55% sweetener by weight of the palatable chewable composition, and 2% to 20% of an encapsulated primary ingredients selected from plant extracts, fiber, vitamins, minerals, and active pharmaceutical ingredients by weight of the palatable chewable composition, wherein at least one of the primary ingredients has a primary coating and a secondary coating, one of which is adapted for masking of odor or flavor, and the other is adapted to serve as a release regulating coating layer.

### Maximum Shear, Temperature, and Moisture/Water Activity

The manufacturing conditions (e.g. maximum shear) may impact the coatings. When mixing with an impeller, peak shear will generally occur near the tips of the impeller where maximum velocity occurs. Velocity is proportional to the rotation rate N (revolutions/sec) multiplied by the impeller diameter D. The total power P applied to fluid being mixed can often be described as P=ρ*N³*D⁵*Kp, where ρ is the specific density of the fluid, N is the rotational speed, D is the impeller diameter, and Kp is the power number. The power number is a parameter that takes into account tank impeller design, fluid viscosity, and Reynolds number. The power density P/V is the power delivered to a given volume V of fluid in the tank, and is related to the energy level being applied to the fluid by the mixer.

The impeller rotational speed is v=π*D*N, and this speed is the source of the maximum shear stress near the impeller tip which is expected to be proportional to the impeller speed, τₘₐₓ~v/D. The physical threats to the integrity of coating layers or the coated actives themselves may be related to power density, τₘₐₓ, or related factors. Measures of coating fragility may be considered such as measures of brittleness or frangibility, mechanical strength, elasticity, or resilience. One or more such measures may need to be increased in response to a design change or raw material change that increases the risk to the encapsulated particle.

When a brittle or frangible coating is desired for compatibility with the active, the palatable chewable composition matrix or the intended release profile, for example, the impeller of a mixing tank may be modified in term of diameter, speed, or design to reduce shear. For example, a sawtooth impeller may be used when high shear is needed, and a hydrofoil and anchor impeller may be used when gentler, low-shear mixing is desired. Of course, impeller speed may be adjusted to regulate shear as well.

The aqueous solution used to make the palatable chewable composition matrix can also pose a threat to some coatings, especially prior to gelling when the activity of the water aw may be high. The solution may also damage or etch the coating due its pH, such as a low pH between about 3 to about 5 due to the presence of citric acid or other acidifiers. When a release control coating material is sensitive to the pH of the matrix and/or its water activity or other ingredients, it may be further isolated from the other materials with a secondary coating such as a shellac, a wax with an elevated melting point, various polymers to provide protection.

Elevated temperature may also be avoided for some actives and for some release control coatings, which may be damaged (softened or melted, for example, or caused to engage in an adverse reaction with other materials or itself) by elevated temperatures. Secondary coatings such as a wax with an elevated melting point of shellac may be used in such cases, or an alternate formulation for the primary coating may be used. Thus, the one or more layers of a coating may be selected to protect the active and the release control layer on the active in designing each encapsulated primary ingredient to achieve good results in manufacturing, storage during the shelf life of the product, and after ingested.

### Palatable Chewable Composition Product Analysis

The palatability of the palatable chewable composition may be determined by a Descriptive Analysis Panel.

A Descriptive Analysis Panel is a structured sensory evaluation methodology in which a group of trained human assessors generate a consensus profile of the organoleptic attributes of a composition. Panelists are trained to identify, quantify, and agree upon the presence and intensity of specific sensory attributes such as bitterness, sweetness, mouthfeel, and aftertaste. Unlike instrumental methods such as electronic tongue (E-tongue) analysis, which rely on arrays of chemical sensors to generate objective electrochemical fingerprints, the Descriptive Analysis Panel captures the human sensory experience directly, relying on trained consensus scoring to reduce variability and ensure reproducibility. Accordingly, the Descriptive Analysis Panel provides qualitative and quantitative information on palatability that complements instrumental E-tongue data and allows for confirmation that a composition achieves improved patient acceptability. Descriptive Analysis Panels are generally known in the art.

### Packaging

The palatable chewable composition may be packaged in a container including other palatable chewable compositions having a variety of flavors and colors. Such containers may include plastic bottles sealed with a foil layer or other relatively oxygen-impermeable material. When there is a risk of flavors and aromas migrating from one palatable chewable composition to another, palatable chewable compositions may be segregated if desired into smaller units of same-flavor or compatible flavor groupings. Blister packs or single-dose packaging formats may be used in such cases. Blister packs, for example, may be made of a plastic sheet molded to have a matrix or series of blisters that can receive one or more palatable chewable compositions, and then be sealed with a foil layer or other relatively oxygen-impermeable layer (e.g., a paper web treated with a suitable oxygen barrier coating such as EVOH, PVOH, or an vapor-deposited treatment of aluminum or alumina on a polymer or the paper itself).

Palatable chewable compositions or small numbers of palatable chewable compositions (e.g., 2 or 3) may also be individually wrapped. In general, any suitable packaging from unit dose to large containers may be considered.

### Method of Use

Further provided herein are methods of delivering one or more active pharmaceutical ingredients to a subject in need thereof. In particular embodiments, the method may comprise administering a palatable chewable composition as described herein to a subject in need thereof.

The palatable chewable composition used in these methods includes a primary ingredient that is an active pharmaceutical ingredient. Any active pharmaceutical ingredient may be used in the palatable chewable compositions for delivering one or more therapeutic agent. In some embodiments, the primary ingredient comprises at least one analgesic and at least one antipyretic active pharmaceutical ingredient. In some embodiments, analgesic and antipyretic functions may be provided by the same active pharmaceutical ingredient, while in other embodiments, separate active pharmaceutical ingredients may be included to provide the combined therapeutic effect.

In certain embodiments, the subject may be at least 6 years of age. In pediatric embodiments, dosing may be adjusted based on body weight or age, such as administering from about 5 mg/kg to about 15 mg/kg acetaminophen every 4 to 6 hours, or about 7 mg/kg to about 10 mg/kg ibuprofen every 6 to 8 hours, as clinically indicated. In adult embodiments, the chewable composition may deliver standard over-the-counter unit doses, such as 325 mg to 500 mg acetaminophen per chew, or 200 mg ibuprofen per chew.

The method may be used to treat or reduce fever associated with viral or bacterial infections, immunization reactions, or other febrile conditions, as well as to alleviate mild to moderate pain associated with headache, sore throat, muscle pain, dental pain, arthritis, or other conditions responsive to analgesic/antipyretic therapy. The chewable composition may be administered as a single unit or multiple units depending on the desired therapeutic effect, the severity of the condition, and the age and weight of the subject.

### TERMS AND DEFINITIONS

When introducing elements of various aspects described herein, the articles “a,” “an,” “the,” and “said” are intended to mean that there are one or more of the elements, and thus may include plural referents unless the context clearly dictates otherwise. The terms “comprising,” “including,” and “having” are intended to be inclusive and mean that there may be additional elements other than the listed elements.

As used herein, the word “exemplary” means serving as an example, instance, or illustration. The aspects described herein are not limiting but rather are exemplary only. It should be understood that the described aspects are not necessarily to be construed as preferred or advantageous over other aspects. Unless otherwise indicated, no aspect described herein should be assumed to have the same advantages or features had by any other aspect.

As used herein, the term “palatable” or “palatability” refers to the organoleptic acceptability of a drug formulation by a subject, encompassing sensory attributes such as taste, odor, mouthfeel, and aftertaste. In oral compositions, palatability is a key determinant of patient compliance, particularly in pediatric and geriatric populations. Palatability may be enhanced through the use of flavoring agents, sweeteners, taste-masking techniques, or formulation strategies that reduce or modify the perception of undesirable drug characteristics. Palatable refers to wide acceptance to consume the palatable chewable composition contributed to an aspect of its texture and taste. This can include making the palatable chewable composition easier to swallow via size, shape, coating and texture. A composition is palatable when it successfully masks bitterness or provides a more appealing texture.

As used herein, the term “chewable composition” refers to a type of oral delivery system designed to be chewed but could be swallowed without chewing, providing an active pharmaceutical ingredient or excipient (such as vitamins, minerals or supplements) with additional health benefits. A palatable chewable composition not only offers a convenient and palatable composition but also serves a targeted physiological or therapeutic purpose. Examples include but are not limited to gummies, chewable gels, soft chews, or soft chewable tablets.

As used herein, the term “gummy” refers to a broad confectionery product type comprised of a hydrocolloid gelling agent, such as pectin, carrageenan, gelatin, etc., a boiled mixture of mixed carbohydrates, and about 12%-24% by weight of moisture. A gummy is typically soft and chewy, but can have a wide range of textures deriving from the specific nature of the formulation and manufacturing process.

As used herein, the term “pastille” refers to a type of gummy that is prepared using gum Arabic. In a pastille, the gum Arabic typically comprises up to 50% by weight of the solid composition, and produces a hard, short, somewhat malleable texture. The use of gelatin may sometimes be employed to create a softer, longer texture. Pastilles are normally prepared from gum Arabic alone which is the major ingredient constituting some 50% of all total solid matter that is present. The texture produced is hard and short, but malleable. If the level of gum Arabic is reduced to produce a softer eating product then another gelling agent such as gelatine will be required.

As used herein, the terms “chewable gel” or “chewable jelly” refer to a type of gummy typically comprising a hydrocolloid that can also be used to form a jelly or jam. This is typically pectin, carrageenan, or agar. The resulting product is non-flowable with a defined shape.

As used herein, the term “chewable bar” refers to a further defined type of gummy or chewable gel that has been formed into a regular geometric shape, typically a disc or rectangular prism.

As used herein, the term “soft chewable tablet” refers to tablets that are typically made by a molding or extrusion process, frequently with more than 10% by weight water to help maintain a pliable, soft product. Furthermore, a soft chewable tablet comprises attributes of a chewable tablet drug dosage form, which should be easy to chew, palatable, of appropriate size and shape, and able to disintegrate readily to facilitate dissolution.

As used herein, the term “panned chew” refers to a confectionery product type comprising a hard coating surrounding a chewy center. The coating may typically be made via hard panning, which involves the use of sequential application of layers of saturated engrossing syrups. The chewy center typically has low moisture with or without the inclusion of fat.

As used herein, the term “soft chew” refers to a confectionery product type comprising low moisture (less than 10%) and a syrup bulking-ingredient base. Most ingredients are low-moisture, which allows the chew to be made with or without heating. The resulting product is typically soft and pliable but may have a range of textures deriving from the specific nature of the formulation and manufacturing process. As an example, a soft chew could be extruded in order to produce a defined shape and size prior to packaging.

As used herein, the term “primary ingredient” refers to an active pharmaceutical ingredient, a dietary supplement, a food, and any combination thereof.

As used herein, the term “active pharmaceutical ingredients” or “active ingredient(s)” or “drug” refers to any therapeutically active agent. The term includes any therapeutic active agent indicated for oral administration, such as NSAIDs, antibiotics, anticoagulants and antiplatelet agents, antidiabetic agents, antiemetics, antihistamines, anti-asthmatic medications, antiarrhythmics, anticholinergics, anticholinesterase inhibitors, anticonvulsants, antidepressants, antidiabetic agents, antihistamines, antihypertensives (ACE inhibitors, alpha blockers, angiotensin II receptor blockers, beta blockers, calcium channel blockers, central alpha agonists, diuretics, and the like), anti-inflammatory drugs, antipsychotics, antipyretics, appetite suppressants, bronchodilators, chemotherapy compounds, cholesterol level medications (bile acid sequestrants, cholesterol absorption inhibitor such as ezetimibe, fibrates, PCSK9 inhibitors, statins, angiotensin II receptor blockers, beta blockers, calcium channel blockers, central alpha agonists, and the like), diuretics, expectorants, hormonal medications, laxatives, sedatives, stimulants, vasodilators, anxiolytics, mood stabilizers, antipsychotics, antidepressants and antipsychotics, and the like. The term may refer to more than one therapeutic agent, wherein exemplary combinations of therapeutic agents include a combination of two or more. The term also includes but is not limited to, minerals, vitamins, nutraceutical agents, and other supplements; including derivatives, salts (and the like), and/or mixtures of the foregoing.

As used herein, the term “analgesic” refers to a drug or substance that relieves pain without causing loss of consciousness.

As used herein, the term “antipyretic” refers to a drug or substance that reduces fever by lowering an elevated body temperature.

As used herein, the term “hydrocolloid” refers to a hydrophilic polymeric substance, typically of natural, semi-synthetic, or synthetic origin, that disperses in water to form a viscous solution, gel, or film. Hydrocolloids include but are not limited to polysaccharides (e.g., pectin, alginates, xanthan gum, cellulose derivatives) and proteins (e.g., gelatin), and they may function as thickeners, stabilizers, film formers, or gelling agents depending on the formulation context.

As used herein, the term “gelling agent” refers to an excipient intentionally incorporated into a pharmaceutical formulation to create a gel, that is, a semi-solid system in which a liquid phase is immobilized within a three-dimensional network. Gelling agents may be hydrocolloids (e.g., pectin, carbomers, cellulose derivatives) or non-hydrocolloids (e.g., silica, bentonite), but are distinguished by their functional role of producing gel structures in the final composition. Gelling agents may provide viscosity and stability to compositions.

As used herein, the term “encapsulation” refers to the action of enclosing, surrounding, complexing, intertwining, comingling one object by another or as if in a capsule, which may be exemplified by, but not limited to, cyclodextrins, ion-exchange resins, liposomes, micelles, microencapsulation, spray drying, or coacervation and other techniques known by those skilled in the art.

As used herein, the term “shelf life” refers to the specific time period during which a final product meets and maintains all quality specifications and remains suitable for consumers. This period is dependent on defined environmental conditions, including factors such as temperature, relative humidity, and packaging configuration, which must be controlled to preserve the product's quality and stability.

As used herein, the term “humectant” refers to any pharmaceutically, cosmetically, nutraceutically, or food-acceptable substance that is hygroscopic, meaning it can attract, bind, and retain water molecules from its surroundings or from an underlying substrate. By doing so, the substance increases or maintains the water content, plasticity, softness, or flow characteristics of a composition, article, or biological tissue. Suitable humectants include but are not limited to polyols such as glycerin, propylene glycol, butylene glycol, sorbitol, and xylitol; low-molecular-weight polyethylene glycols such as PEG-300 to PEG-600; sugars and sugar syrups such as glucose syrup, inverted sugar syrup, and honey; urea; betaine; sodium pyrrolidone carboxylate (sodium PCA); lactic acid or its salts; hyaluronic acid and its salts; ethoxydiglycol; and mixtures thereof. Unless expressly excluded, the term also encompasses natural, synthetic, and chemically modified derivatives, provided the substance exhibits sufficient water-binding capacity to measurably reduce water loss or increase the water activity of the formulation in which it is employed.

As used herein, the term “pharmaceutical performance” refers to the ability of a composition product to deliver its active pharmaceutical ingredients effectively and consistently to achieve the desired therapeutic effect. This includes characteristics such as bioavailability, the rate and extent of API release, uniformity of dosage, disintegration time, stability, palatability, and ease of administration, all of which contribute to the efficacy, safety, and user acceptability of the product.

As used herein, “suitable release profile” refers to, but is not limited to instant release profile or immediate release profile, sustained release profile or extended release profile, and delayed release profile. The release profile is effected by the drug and/or drug formulation to be administered (e.g., coatings, specific excipients, and other drug product attributes), the concentration of drug in the formulation, the medium for which the release or dissolution of the product takes place, and a variety of other factors that are appreciated by those of ordinary skill in the art.

As used herein, an “instant release profile” or an “immediate release profile” refers to a release profile wherein no less than about 80% of the API is released in 60 minutes in vitro as measured in a USP approved Type 2 paddle apparatus at 50 rpm or 100 rpm at a constant temperature of 37±0.5° C. in dissolution media with pH between 1.2 to 6.8.

As used herein, a “sustained release profile” or an “extended release profile” refers to a release profile wherein no more than about 80% of the API is released within about 6 hours in vitro as measured in a USP approved Type 2 paddle apparatus at 50 rpm or 100 rpm at a constant temperature of 37±0.5° C.

As used herein, a “delayed release profile” refers to a release profile wherein, dependent upon the number of units tested, the average cannot exceed 10% and no individual unit can exceed 25% of the API released during the acid stage, performed using 0.1 N HCL for 2 hours, as tested in vitro and measured in a USP approved apparatus at a constant temperature of 37±0.5° C. After completion of the acid stage, each unit should have, at least, 75% of the initial API dose remaining and available for release in a USP buffer media, pH 4.5-7.5. During the buffered stage, a multipoint dissolution profile should be obtained using adequate sampling times such as, but not limited to, 15, 30, 45, 60, and 120 minutes, resulting in the release of no less than 80% of the initial, pre acid stage, API dose at a constant temperature of 37±0.5° C.

As used herein, “taste-masking agents” refer to any materials, compounds, or formulation components that reduce, suppress, block, overwhelm, counteract, or otherwise diminish undesirable sensory attributes associated with an active ingredient, food ingredient, or excipient. Undesirable sensory attributes may include bitterness, astringency, metallic notes, sulfury notes, chemical off-flavors, medicinal or phenolic notes, heat or burn sensations, acidity, sourness, or lingering aftertaste.

As used herein, “efficacy” refers to the ability of a formulation, ingredient, encapsulate, or taste-masking system to achieve its intended functional purpose. The functional purpose includes but is not limited to reducing undesirable sensory attributes, preserving active ingredient integrity during processing, delivering controlled release, improving palatability, or maintaining acceptable initial-taste and after-taste profiles.

As used herein, “about” refers to +10% of the stated numerical value. In other embodiments, depending on the context and precision of the measurement, “about” may refer to +5%, +2%, +1%, or +0.5% of the stated value. A value preceded by “about” therefore includes the recited value itself and encompasses values within the range that do not materially alter the performance, characteristics, or function of the invention.

### EXAMPLES

### Example 1: Acetaminophen Chewable Compositions—Preparation and Characterization

A series of acetaminophen-containing chewable compositions were prepared to evaluate the effect of acetaminophen concentration, encapsulation, and taste-masking agents on sensory characteristics. Matrices were prepared using a pectin-based system. Acetaminophen (APAP) was incorporated at concentrations corresponding to 10%, 20%, 30%, 40%, 50%, 60%, and 70% of a 325-mg label claim per 5 gram piece (Samples 1-7). Two samples (325 mg APAP per 5-gram piece) were prepared using encapsulated acetaminophen without taste maskers (Sample 8) or with taste maskers (Sample 9).

The compositions are summarized in Table 1.

TABLE 1

| | % | APAP | | |
|---|---|---|---|---|
| Sample | APAP | mg/5 g | Encapsulated | Maskers |
| Sample 1 | 10% | 32.5 | No | No |
| Sample 2 | 20% | 65 | No | No |
| Sample 3 | 30% | 97.5 | No | No |
| Sample 4 | 40% | 130 | No | No |
| Sample 5 | 50% | 162.5 | No | No |
| Sample 6 | 60% | 195 | No | No |
| Sample 7 | 70% | 227.5 | No | No |
| Sample 8 | 100% | 325 | Yes | No |
| Sample 9 | 100% | 325 | Yes | Yes |

### Descriptive Sensory Analysis

A sensory evaluation was conducted using a trained Descriptive Analysis (DA) Panel to quantify flavor, taste, and chemical-feeling-factor attributes of the disclosed compositions. The procedure followed the methodology set forth in Sensory Evaluation Techniques, (Carr, Civille, Meilgaard, Osdoba, 2025, 6th Ed., CRC Press), the entire contents of which are incorporated herein by reference.

Panelists were screened to ensure normal olfactory and gustatory function and were excluded if they exhibited temporary or chronic sensory impairment, including colds, allergies, anosmia, taste disorders, or other conditions that may interfere with sensory reliability. Selected panelists underwent calibration training for the target attributes and were instructed on use of the 15-point Spectrum Scale, where 0 denotes none and 15 denotes very strong intensity.

Calibration references included: 1) Bitterness: caffeine solutions at 0.05%, 0.08%, 0.15%, and 0.20% (w/v), corresponding to Spectrum intensities 2, 5, 10, and 15; 2) Sweetness: sucrose solutions at 2%, 5%, 10%, and 16% (w/v), corresponding to Spectrum intensities 2, 5, 10, and 15; and 3) Mouth burn: black pepper, white pepper, or cayenne pepper solutions (used exclusively for ibuprofen-containing samples).

All samples were presented blind using randomized three-digit codes, and all evaluations were conducted simultaneously across panelists to maintain synchronized timing. A minimum of six trained panelists participated in each session. Between samples, a mandatory 10-15 minute inter-stimulus interval was enforced. Room-temperature water and unsalted crackers were provided for palate cleansing. All samples were evaluated in replicate, and mean intensity scores were recorded for analysis.

Each sample was evaluated over a 45-second sensory window. Initial Taste was assessed after 15 seconds of mastication, immediately prior to expectoration. Aftertaste was assessed 30 seconds after expectoration, corresponding to 45 seconds from initial chew onset.

The DA panel evaluated both initial and aftertaste intensities for the following attributes: mixed berry flavor, sweetness, bitterness, medicinal (a subjective assessment based on panelist consensus regarding chemical or medicinal notes (not formally calibrated)), and mouth burn (for ibuprofen only).

Bitterness was calibrated using caffeine reference solutions. A Spectrum Scale intensity score of 2 corresponded to a 0.05% (w/v) caffeine solution, a score of 5 corresponded to 0.08% caffeine, a score of 10 corresponded to 0.15% caffeine, and a score of 15 corresponded to 0.20% caffeine.

Sweetness was calibrated using sucrose solutions. A Spectrum Scale intensity score of 2 corresponded to a 2.0% (w/v) sucrose solution, a score of 5 corresponded to 5.0% sucrose, a score of 10 corresponded to 10% sucrose, and a score of 15 corresponded to 16% sucrose.

### Bitterness

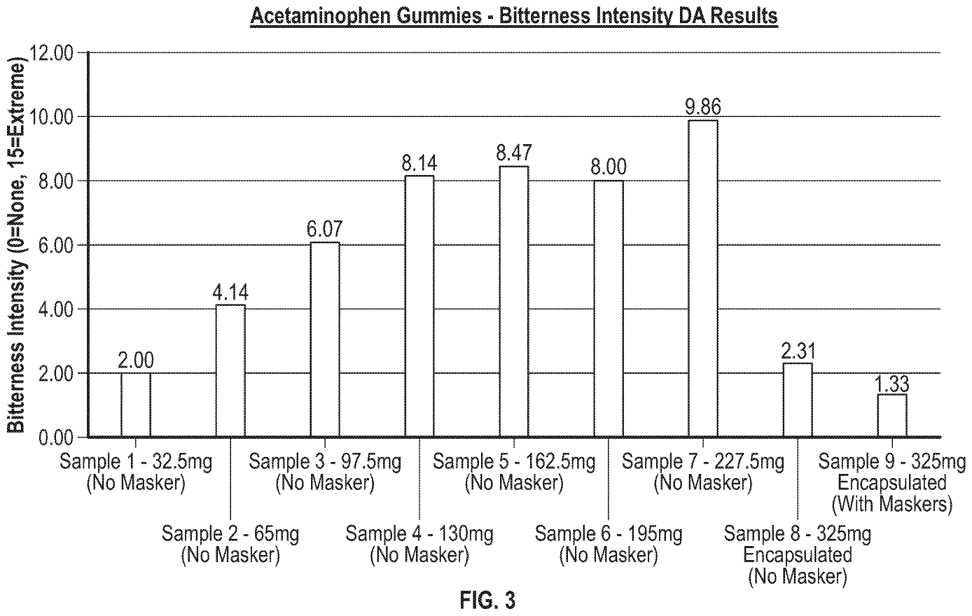

As shown in FIG. 3, the bitterness intensity increased with acetaminophen concentration among non-encapsulated chewable compositions. The mean bitterness values for Samples 1-7 were 2.00, 4.14, 6.07, 8.14, 8.47, 8.00, and 9.86, respectively.

Encapsulation substantially decreased bitterness. Sample 8 (encapsulated, 325 mg APAP) exhibited a mean bitterness intensity of 2.31. Sample 9 (encapsulated, 325 mg APAP, with maskers) exhibited a bitterness intensity of 1.33. Thus, encapsulation alone reduced bitterness by approximately 72%, and the combination of encapsulation+maskers reduced bitterness by approximately 86% relative to high-dose non-encapsulated chewable compositions.

### Sweetness

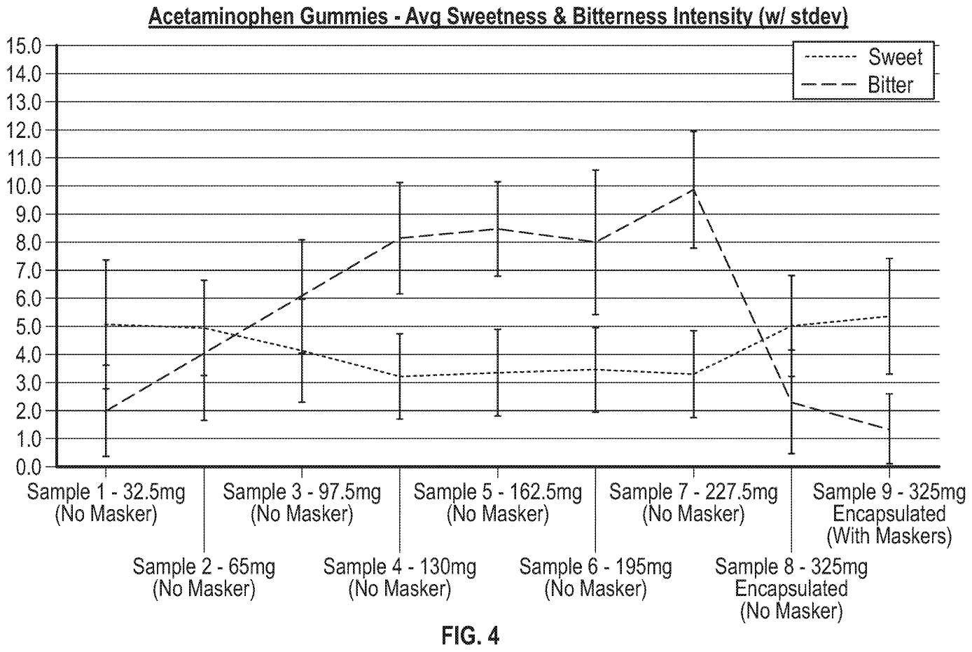

As shown in FIG. 4, sweetness values for Samples 1-7 remained between approximately 3.21 and 5.07 and did not exhibit a proportional increase with APAP concentration. Samples 8 and 9 showed sweetness intensities of 5.00 and 5.36, respectively.

### Example 2: Comparison of Soft Chewable Tablet Acetaminophen Compositions with Commercially Available Products

Chewable acetaminophen compositions were prepared using a traditional pectin-based chewable matrix. Each soft chewable tablet (SCT) weighed approximately 5.2 grams and contained red berry flavoring and a taste-masking agent. Four formulations were prepared, varying in primary ingredient loading. The compositions are summarized in Table 2.

TABLE 2

| | APAP | | | | |
|---|---|---|---|---|---|
| | Content | | | | |
| | (mg per | Additional | | | |
| Sample | 5.2 g) | Actives | Base | Flavor | Masker |
| SCT #1 | 162.5 | None | Pectin | Red | Yes |
| | | | | Berry | |
| SCT #2 | 162.5 | Dextrome- | Pectin | Red | Yes |
| | | thorphan | | Berry | |
| | | HBr (5 mg) | | | |
| SCT #3 | 325 | None | Pectin | Red | Yes |
| | | | | Berry | |
| SCT #4 | 500 | None | Pectin | Red | Yes |
| | | | | Berry | |

The formulations were manufactured by heating and dissolving the pectin and sweeteners, incorporating the flavor and taste masker, and blending with acetaminophen and optional Dextromethorphan HBr. The mixture was deposited into molds and cooled to form soft chewable tablets.

### Sensory Evaluation and Descriptive Analysis

A trained descriptive sensory panel evaluated flavor attributes of the compositions described in Table 2. Testing was conducted according to the descriptive analysis protocol described in Example 1. Sensory intensities were evaluated on a 15-point Spectrum scale, where 0 indicates none and 15 indicates very strong. Each sample was evaluated in duplicate.

Attributes evaluated included fruity flavor, medicinal character, basic taste sweetness, and basic taste bitterness. Initial intensity was evaluated after a 15-second chew for soft chewable tablets and a 5-second swish for comparator liquids. Aftertaste intensities were measured at 30 seconds post-expectoration.

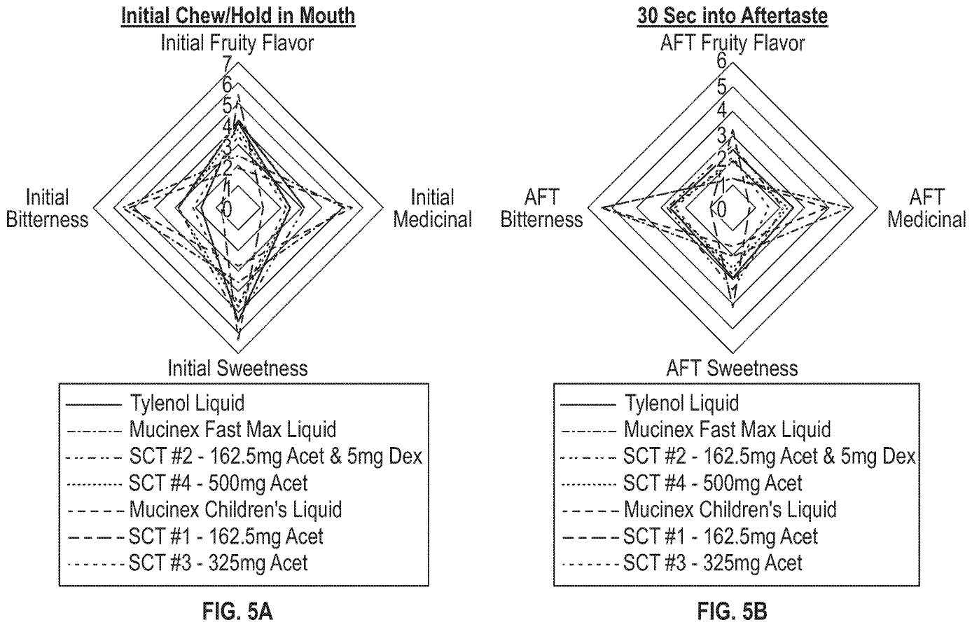

Comparator samples included three commercial acetaminophen-containing liquids: Tylenol® Children's & Adults Liquid (160 mg/5 ml), Mucinex® Children's Cold & Flu Liquid (325 mg APAP+additional APIs), and Mucinex® Fast-Max Cold & Flu Liquid (650 mg APAP+additional APIs). The results are presented in Table 3 below and in the plots in FIG. 5A and FIG. 5B.

TABLE 3

| | Sample Identifier | | | | | | |
|---|---|---|---|---|---|---|---|
| | | | | | 5 | | |
| | | 2 | 3 | 4 | SCT #2- | 6 | 7 |
| | 1 | Mucinex | Mucinex | SCT #1- | 162.5 mg | SCT #3 | SCT #4- |
| | Tylenol | Children's | Fast Max | 162.5 mg | APAP & | 325 mg | 500 mg |
| | Liquid | Liquid | Liquid | APAP | 5 mg Dex | APAP | APAP |
| Initial | 4.1 | 1.9 | 2.5 | 5.4 | 4.2 | 3.5 | 3.8 |
| Fruity | ab | d | cd | a | ab | bc | bc |
| Flavor | 3, 2 | 4, 5, 1, 7, 6 | 4, 5, 1 | 7, 6, 3, 2 | 3, 2 | 4, 2 | 4, 2 |
| Initial | 2.5 | 5.5 | 5.1 | 1.2 | 3.2 | 2.3 | 2.5 |
| Medicinal | bc | a | a | c | b | bc | bc |
| | 2, 3 | 5, 1, 7, 6, 4 | 5, 1, 7, 6, 4 | 2, 3, 5 | 2, 3, 4 | 2, 3 | 2, 3 |
| Initial | 5.5 | 2.8 | 3.6 | 6.3 | 5.4 | 4.7 | 4.8 |
| Sweetness | ab | d | cd | a | ab | bc | bc |
| | 3, 2 | 4, 1, 5, 7, 6 | 4, 1, 5 | 7, 6, 3, 2 | 3, 2 | 4, 2 | 4, 2 |
| Initial | 1.8 | 5.3 | 5.6 | 0.8 | 2.9 | 2.2 | 2.9 |
| Bitterness | bc | a | a | c | b | bc | b |
| | 3, 2 | 5, 7, 6, 1, 4 | 5, 7, 6, 1, 4 | 3, 2, 5, 7 | 3, 2, 4 | 3, 2 | 3, 2, 4 |
| AFT | 2.4 | 1.2 | 1.2 | 3.2 | 2.6 | 2.1 | 1.9 |
| Fruity | abc | c | c | a | ab | abc | bc |
| Flavor | | 4, 5 | 4, 5 | 7, 2, 3 | 2, 3 | | 4 |
| AFT | 2.5 | 3.9 | 4.8 | 0.7 | 2.1 | 1.5 | 2.3 |
| Medicinal | b | a | a | c | bc | bc | b |
| | 3, 2, 4 | 1, 7, 5, 6, 4 | 1, 7, 5, 6, 4 | 3, 2, 1, 7 | 3, 2 | 3, 2 | 3, 2, 4 |
| AFT | 2.9 | 1.6 | 2 | 4.1 | 3.4 | 2.8 | 2.5 |
| Sweetness | abc | d | cd | a | ab | bcd | bcd |
| | | 4, 5, 1 | 4, 5 | 6, 7, 3, 2 | 3, 2 | 4 | 4 |
| AFT | 2.5 | 5.4 | 5.3 | 0.9 | 2.7 | 2.4 | 2.6 |
| Bitterness | bc | a | a | c | b | bc | bc |
| | 2, 3 | 5, 7, 1, 6, 4 | 5, 7, 1, 6, 4 | 2, 3, 5 | 2, 3, 4 | 2, 3 | 2, 3 |

### Initial Flavor and Taste Characteristics

Initial intensities for fruity flavor, medicinal notes, sweetness, and bitterness were determined for each sample. FIG. 5A is a graphical representation of the results.

Fruity flavor: SCT #1 exhibited the highest initial fruity flavor intensity (5.4). All SCT samples demonstrated fruity flavor intensities between 3.5 and 5.4. In contrast, the tested liquid products exhibited a statistically significant (p<0.05) lower fruity flavor (1.9-4.1).

Medicinal Character: Commercial liquids demonstrated elevated medicinal character (5.1-5.5). The SCT compositions demonstrated substantially lower medicinal intensity (1.2-3.2).

Sweetness: SCT formulations demonstrated sweetness intensities between 4.7 and 6.3, exceeding or matching those of commercial liquids (2.8-5.5).

Bitterness: Initial bitterness intensities were 0.8-2.9 for SCT samples and 5.3-5.6 for the Mucinex liquids. Tylenol liquid exhibited an intensity of 1.8.

These results demonstrate that the inventive soft chewable formulations provide significantly reduced bitterness and medicinal notes relative to commercial APAP liquids.

### Aftertaste Characteristics

Aftertaste intensities were evaluated 30 seconds after expectoration. FIG. 5B is a graphical representation of the results.

Fruity Flavor: SCT samples maintained fruity aftertaste intensities between 1.9 and 3.2, generally higher than those of the liquid products (1.2-2.4).

Medicinal Character: Aftertaste medicinal intensities for SCTs were between 0.7 and 2.3. Liquid products remained higher, with values of 3.9 and 4.8 for the Mucinex products.

Sweetness: SCTs exhibited sweetness intensities of 2.5-4.1 at the aftertaste time point, while liquids exhibited values of 1.6-2.9.

Bitterness: Aftertaste bitterness for SCTs ranged from 0.9 to 2.7. Mucinex liquids exhibited aftertaste bitterness of 5.3-5.4.

These data demonstrate that the soft chewable tablet maintains low bitterness and medicinal character not only during initial evaluation but throughout the aftertaste period.

### Effect of Active Load on Sensory Performance

As shown in Table 3 and FIGS. 5A and 5B, soft chew formulations containing 162.5 mg, 325 mg, and 500 mg acetaminophen (SCT #1, #3, and #4) were evaluated to determine the impact of active loading on sensory perception.

Across all three acetaminophen loadings, bitterness remained low (initial: 0.8-2.9; aftertaste: 0.9-2.7), fruity flavor remained moderate to high (initial: 3.5-5.4; aftertaste: 1.9-3.2), and medicinal notes remained low (initial: 1.2-3.2; aftertaste: 0.7-2.3)

These results indicate that increasing acetaminophen content up to 500 mg per 5.2 g chew did not substantially increase bitterness or medicinal character when formulated in a pectin chew base with flavor and taste masker.

### Comparison to Commercial Acetaminophen Liquids

Relative to commercial liquids containing equivalent or higher levels of acetaminophen, the inventive soft chewable tablet formulations had significantly lower bitterness, significantly lower medicinal character, higher fruity flavor, higher sweetness, and improved aftertaste profile. Surprisingly, the sensory performance of the soft chewable tablets was superior across all tested attributes, irrespective of acetaminophen concentration.

The content uniformity of APAP was demonstrated in a 5.0 g piece weight, each containing 162.5 mg of APAP. The average APAP content was 164.23 mg/piece with standard deviation of 8.00 mg.

The formulation achieves dissolution profile that fulfills the requirements of using method listed in USP Chapter <711>.

### Example 3: Preparation of the Inventive Encapsulated Acetaminophen Compositions

### Process Parameters

A design of experiments (DOE) evaluation was conducted using the Heidolph reactor system to characterize the processing behavior of encapsulated acetaminophen (APAP). Encapsulated APAP was introduced into multiple confectionery base systems, including sucrose/corn syrup systems, pectin matrices, and gelatin matrices.

The four major factors identified were (1) temperature, (2) shear applied within the mixer, (3) residence time within the depositor hopper or heated zones, and (4) the base solvent system.

Across all experiments, the encapsulated APAP compositions were processed under controlled thermal conditions ranging from approximately 70° C. to 95° C., with agitation provided by a 1 L jacketed glass reactor. The goal was to assess the robustness of the encapsulate under processing conditions representative of eventual line-scale manufacturing.

### Temperature and Shear Effects on Encapsulate Integrity

Temperature effects were evaluated by exposing the sugar/corn syrup base systems to controlled heating between 75° C. and 85° C. Survival of the encapsulate remained above 90%, indicating excellent thermal stability in this range.

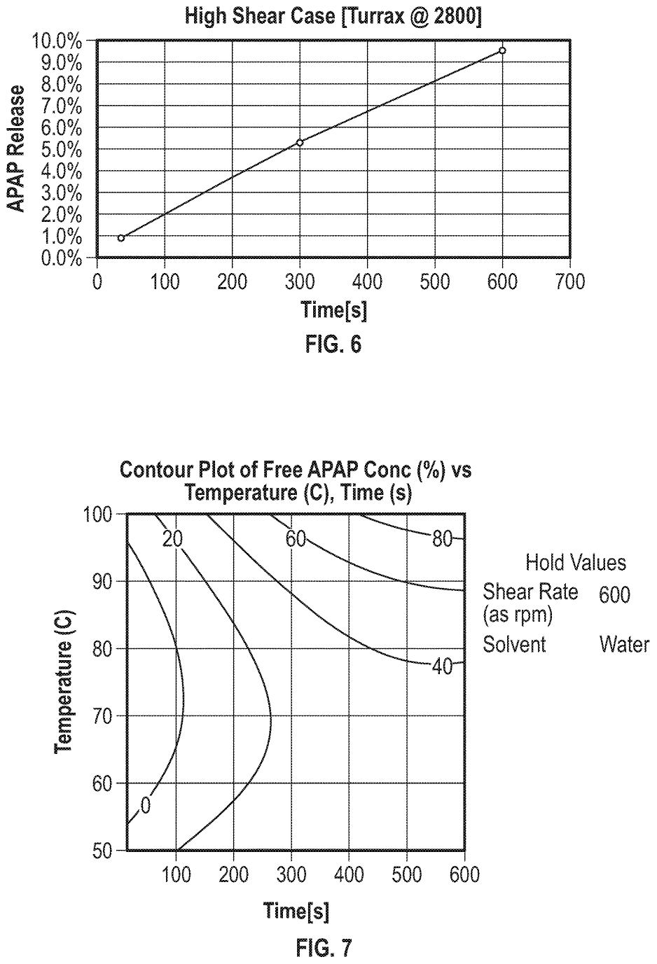

Shear effects were investigated by subjecting the sugar/corn syrup base to approximately 3×the shear of the mixer at 85° C. Under these conditions, the encapsulated APAP exhibited only limited release, approximately 10%, which was considered a worst-case scenario. Because the shear levels encountered during normal production are lower and of shorter duration, mixer shear was determined unlikely to cause catastrophic encapsulate failure. The results are illustrated in FIG. 6. As shown, elevated shear exposure increases APAP release over time, with values rising from about 1% at 60 seconds to about 9% at 600 seconds.

Table 4 demonstrates that temperature remains the dominant factor affecting encapsulate integrity, with shear having secondary influence.

TABLE 4

| | | | | Shear | | | |
|---|---|---|---|---|---|---|---|
| | | | | rate | | APAP | |
| Run | Time | Temp | Solids | (as | Viscosity | Concentration | |
| Order | (s) | (° C.) | (%) | rmp) | (cP) | (%) | |
| 15 | 600 | 110 | 80 | 1000 | 364.1 | 69.0 | Temp |
| | | | | | (5.9% | | Impact |
| | | | | | torque) | | |
| | | | | | 201.0 F. | | |
| 20C | 600 | 85 | | 2800 | 527.4 | 9.5 | |
| | | | | | (8.7%) | | |
| | | | | | 173.1 F. | | |
| 18 | 600 | 75 | | 200 | 662.2 | 3.6 | |
| | | | | | (11.0% | | |
| | | | | | torque) | | |
| | | | | | 115.1 F. | | |
| 16 | 600 | 50 | | 1000 | 2077 | 0.9 | |
| | | | | | (34.5% | | |
| | | | | | torque) | | |
| | | | | | 115.7 F. | | |
| 20B | 300 | 85 | | 2800 | 527.4 | 5.3 | Sheet |
| | | | | | (8.7%) | | Impact |
| | | | | | 173.1 F. | | |
| 20A | 35 | 85 | | 2800 | 527.4 | 0.9 | |
| | | | | | (8.7%) | | |
| | | | | | 173.1 F. | | |
| 19 | 600 | 85 | | 200 | 541.3 | 5.9 | |
| | | | | | (9.0% | | |
| | | | | | torque) | | |
| | | | | | 173.4 F. | | |

### Influence of Solids Content, Solvent Composition, and Temperature-Time Profiles

Regression modeling from the DOE screener revealed relationships between solids levels, solvent systems, and APAP release. In water/syrup/sucrose systems, increasing solids content reduced solvent availability and therefore decreased APAP dissolution. When analysis was restricted to the seven aqueous-based DOE runs, solids exhibited the expected negative effect on release. In contrast, in glycerin-containing systems, APAP solubility was higher (approximately 1:40 in glycerin versus 1:70 in water at room temperature). Consequently, increasing solids in glycerin systems increased APAP release, reflecting the greater solvent capability of glycerin.

Temperature-time interactions were also modeled. Maintaining exposure temperatures below 80° C. and minimizing exposure time to under approximately one minute kept free APAP near or below 10%. As a practical rule derived from the DOE, a reduction of approximately 10° C. doubled the allowable holding time before significant APAP release occurred.

### Extended Hold Studies

Extended thermal hold studies were performed using representative slurries including current pectin formulations, gelatin systems, and pectin systems. Samples were held at 85° C.-90° C. for durations of 30-40 minutes. Across all formulations evaluated, free APAP levels remained within the single-digit to low-digit percentage range, indicating strong encapsulate durability even under extended heating.

The inventive formulations, particularly pectin-based formulations, can withstand prolonged processing conditions compatible with commercial confectionery manufacturing.

FIG. 7 is a contour plot showing the concentration of free acetaminophen (APAP) released from an encapsulated acetaminophen as a function of processing temperature (° C.) and time(s) under a fixed shear rate of 600 rpm in a water-based system.

As shown in FIG. 7, the contour regions progress from left to right, corresponding to increasing free APAP levels from <0% up to >60%. At lower temperatures (e.g., 50-60° C.) and short exposure times (<100 seconds), the model predicts minimal APAP release (<5%). As both temperature and time increase, the contour lines shift toward progressively higher release zones.

The plot demonstrates that free APAP concentrations increase in a monotonic fashion with respect to temperature and time. For example, at 80° C., exposure durations exceeding 300-400 seconds transition into higher release regions (30-40% free APAP). At temperatures approaching 100° C., the release exceeds 50%.

This contour mapping provides a visual representation of the thermal-time operating window that maintains APAP release within acceptable limits during the processing of encapsulated inventive composition.

Overall, FIG. 7 demonstrates how reducing shear (600 rpm) expands the allowable processing window and shifts free-APAP contour boundaries toward higher temperature and time thresholds.

### Example 4 APAP Release Profile in Slurry

Five confectionery base formulations were prepared for the evaluation of encapsulated acetaminophen (APAP) release under controlled thermal and shear conditions. The formulations consisted of (i) two gelatin systems, (ii) two pectin systems, and (iii) an exemplary pectin gel chassis system. Table 5 lists the prepared formulation.

TABLE 5

| | Formulation | Temperature | | |
|---|---|---|---|---|
| Run # | Name | (° C.) | Composition | |
| 1 | Gelatin | 65° C. | Water | 18.00% |
| | | | Gelatin | 6.40% |
| | | | Glycerin | 12.80% |
| | | | Corn syrup, 63DE | 6.10% |
| | | | Corn syrup, 43DE | 7.50% |
| | | | Pectin | 0.10% |
| | | | Sodium citrate | 0.00% |
| | | | Palm oil | 8.50% |
| | | | Adare APAP | 6.80% |
| | | | Sucrose | 31.10% |
| | | | Malic acid solution | 2.70% |
| | | | Total: | 100% |
| 2 | Gelatin | 75° C. | Water 1 | 8.00% |
| | | | Gelatin | 6.40% |
| | | | Glycerin | 12.80% |
| | | | Corn syrup, 63DE | 6.10% |
| | | | Corn syrup, 43DE | 7.50% |
| | | | Pectin | 0.10% |
| | | | Sodium citrate | 0.00% |
| | | | Palm oil | 8.50% |
| | | | Adare APAP | 6.80% |
| | | | Sucrose 3 | 1.10% |
| | | | Malic acid solution | 2.70% |
| | | | Total: | 100% |
| 3 | Pectin | 85° C. | Pectin | 1.9% |
| | | | Water | 18.0% |
| | | | Potassium Sodium Tartrate | 0.3% |
| | | | 63DE corn syrup | 34.6% |
| | | | Sucrose | 34.7% |
| | | | Maltodextrin | 0.9% |
| | | | Palm Oil | 1.8% |
| | | | Adare APAP | 7.3% |
| | | | Malic acid | 0.6% |
| | | | Sodium citrate | 0.1% |
| | | | Total | 100.0% |
| 4 | Pectin | 95° C. | Pectin | 1.9% |
| | | | Water | 18.0% |
| | | | Potassium Sodium Tartrate | 0.3% |
| | | | 63DE corn syrup | 34.6% |
| | | | Sucrose | 34.7% |
| | | | Maltodextrin | 0.9% |
| | | | Palm Oil | 1.8% |
| | | | Adare APAP | 7.3% |
| | | | Malic acid | 0.6% |
| | | | Sodium citrate | 0.1% |
| | | | Total | 100.0% |
| 5 | Exemplary | 85° C. | Sucrose | 37.7% |
| | formulation | | 63DE corn syrup | 33.3% |
| | | | Water | 16.8% |
| | | | Encapsulated APAP | 7.0% |
| | | | Pectin | 2.4% |
| | | | Palm Oil | 1.9% |
| | | | Lecithin | 0.5% |
| | | | Buffer Salt | 0.4% |
| | | | Total | 100.0% |

All formulations were performed using a 1 L jacketed glass reactor. Agitation was provided by a Hei-Torque Ultimate 200 stirrer equipped with a 90 mm 4-blade impeller for bulk mixing. Each formulation was subjected to a 1-minute high-intensity mixing period consisting of: 1200 RPM overhead stirring and 2800 RPM Turrax shear.

No further agitation occurred after the initial mixing period. Samples were collected at 1, 10, 20, 30, and 40 minutes. Each sample (15-30 g) was taken from the upper region of the slurry, diluted into 50 g of water, mixed vigorously for ~10 s, and filtered through a 5 μm syringe filter prior to analysis.

Viscosity of each formulation was measured at shear rates of 5-100 RPM. The exemplary pectin gel chassis formulation system with acid did not exhibit significantly higher viscosity than other formulations, despite its more inhomogeneous appearance (Table 6). These results indicate that visual separation behavior is not directly correlated with bulk viscosity (cP) under the measured conditions.

TABLE 6

| | | | | | | Exemplary |
|---|---|---|---|---|---|---|
| | Gelatin | | Pectin | | | Formulation |
| RPM | 65° C. | 75° C. | 75° C. | 84° C. | 94° C. | 84° C. |
| 5 | 12,000 cP | | | | | |
| 10 | | 3,984 cP | 7,920 cP | | | |
| 20 | | 3,648 cP | 7,056 cP | 3,336 cP | 2,592 cP | 3,000 cP |
| 50 | | 3,187 cP | 6,000 cP | 3,187 cP | 1,852 cP | 2,870 cP |
| 100 | | 2,914 cP | | 2,976 cP | 1,598 cP | 2,659 cP |

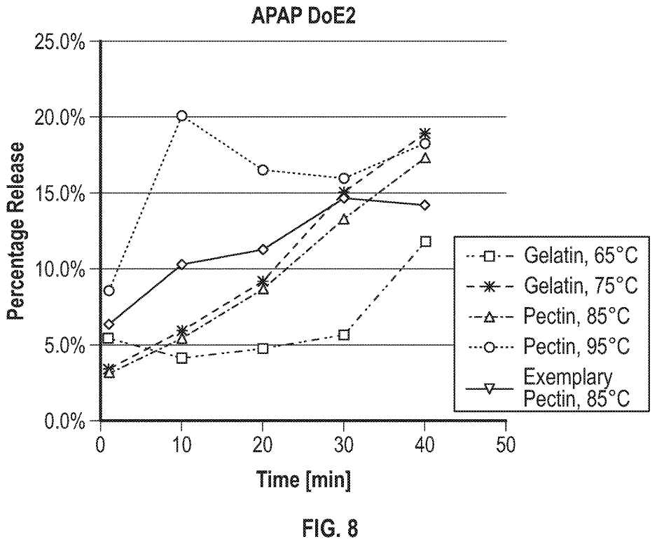

APAP release was quantified over 40 minutes for all five formulations. As shown in the release plot in FIG. 8, all formulations (gelatin (65° C. and 75° C.), pectin (85° C.), pectin with acid (95° C.), and exemplary pectin gel chassis formulation (85° C.)) maintained less than 20% APAP release at 40 minutes, corresponding to <65 mg APAP per 5 g serving, with release increasing as a function of time and processing temperature. This indicates that each matrix, gelatin, pectin (with or without acid), and an exemplary pectin gel chassis formulation, provides adequate protection of the encapsulated APAP under the tested conditions.

Dilution prior to filtration did not cause catastrophic encapsulate rupture but did introduce additional analytical variability compared to standard stress-testing methods.

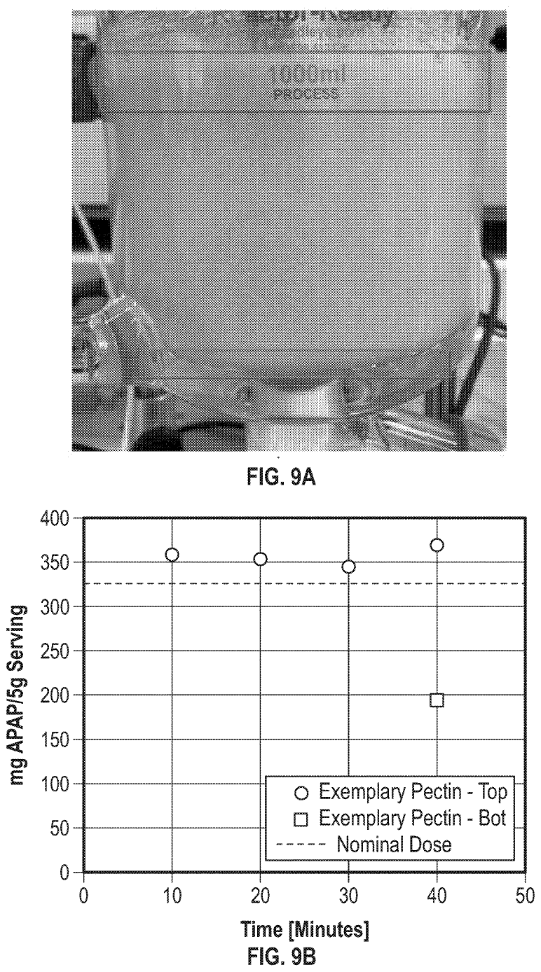

A homogeneity study was conducted on the control pectin formulation by sampling from the top ~1 inch of the slurry and from the bottom of the reactor vessel. FIG. 9A is a photograph showing of the exemplary pectin gel chassis formulation (Run 5, 85° C. with acid) after a 40-minute thermal hold. FIG. 9A shows visual evidence of vertical stratification. The highlighted upper region exhibits a lighter, more dilute phase, while the lower region shows increased particulate density indicative of settling. FIG. 9B is a plot showing APAP assay results from samples collected at the top and bottom of the vessel over a 40-minute hold. Top samples remain near the nominal 325 mg/5 g dose, whereas the bottom sample at 40 minutes shows reduced APAP content, confirming vertical non-uniformity consistent with the observed separation.

### Example 5: Formulations with Caffeine

A method repeatability evaluation was conducted to confirm the robustness of the analytical procedure used to quantify active release in the APAP stress-testing studies. The assessment was performed using a model encapsulated system distinct from acetaminophen, consisting of caffeine coated with an ethyl cellulose dispersion (Surelease Top Spray 23002, 36% coating level). The objective was to determine whether the analytical procedure yields consistent results across replicates under controlled thermal and shear conditions.

The stress test was conducted at 70° C., representing a moderate-temperature environment suitable for evaluating solute permeability through the ethyl cellulose coating. Agitation was supplied using a four-blade impeller at 1000 RPM, corresponding to a medium-shear condition.

Samples were collected at multiple timepoints over a 35-minute interval. Caffeine concentration (mg/g) was quantified for each replicate using the same dilution, agitation, filtration, and assay procedure employed in the primary APAP release studies. This design ensured that method performance could be evaluated independently of variability in encapsulate properties.

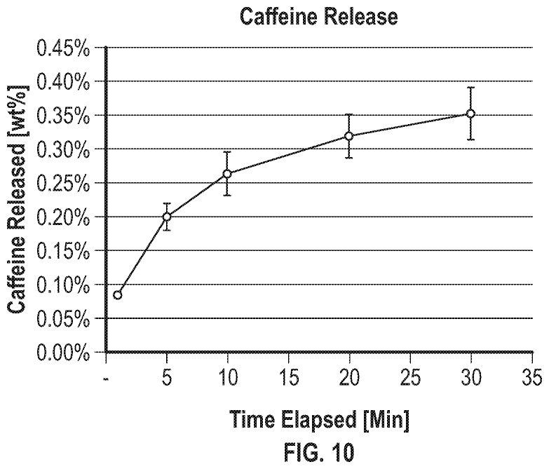

Three independent replicates were analyzed alongside for caffeine. The datapoints and error bars on the graph show the means±standard deviation. As shown in FIG. 10, the standard deviation of caffeine release is low, indicating low batch-to-batch variation.

Ethyl cellulose coatings permit gradual diffusion of soluble actives, consistent with expected permeability properties.

Differences observed in APAP release in Example 4 are therefore attributable to formulation and process effects, not analytical variability.

### Example 6: Preparation of Ibuprofen Soft Chewable Tablets Compositions

Two ibuprofen soft chewable tablet compositions were prepared for sensory evaluation. A control sample contained free (non-encapsulated) ibuprofen, and a test sample contained encapsulated ibuprofen. Both samples were provided to the sensory panel in soft chewable tablet dosage form.

Panelists evaluated flavor and chemical feeling factor attributes using the Descriptive Analysis method described in Example 1. Briefly, trained panelists rate attribute intensity on a 15-point Spectrum Scale (0=none, 15=very strong). All samples were served blind, coded with randomized three-digit identifiers, and evaluated in replicate.

Each panelist chewed a sample for 15 seconds (initial intensity evaluation), expectorated, and then evaluated aftertaste 30 seconds after expectoration (45 seconds total).

The following attributes were measured: fruity flavor, metallic (chemical feeling factor), astringency (chemical feeling factor), mouth burn (chemical feeling factor), and basic taste: sweet or bitter.

Panelists rested for 10-15 minutes between samples, used water and unsalted crackers for palate cleansing, and all evaluations were synchronized to ensure controlled timing.

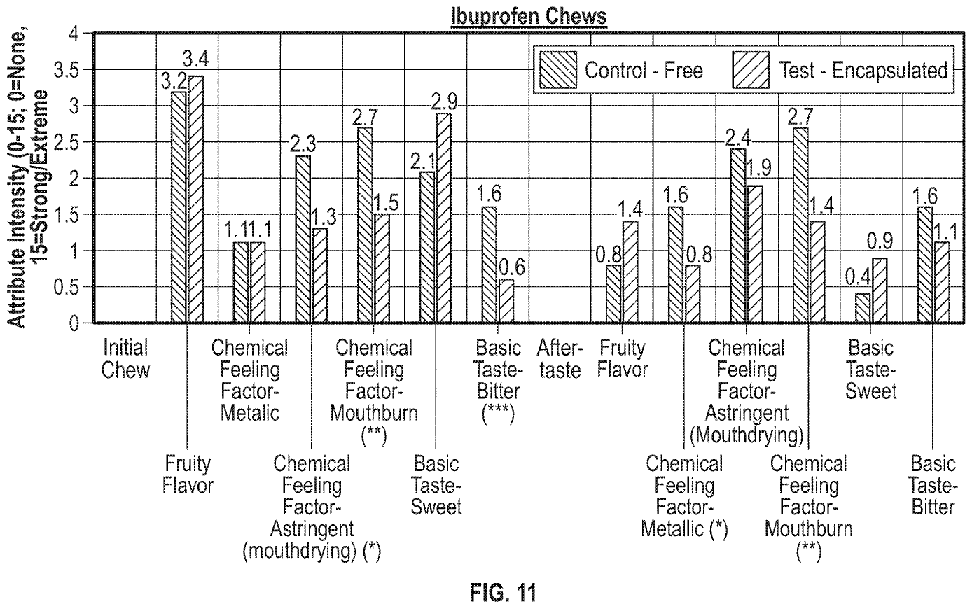

As shown in FIG. 11, the encapsulated ibuprofen soft chewable tablet (Test) demonstrated improved sensory characteristics relative to the free-ibuprofen control: less astringent, weaker mouth burn, and weaker bitterness.

Aftertaste characteristics were evaluated at 30 seconds post-expectoration (45-second total flavor time). As shown in FIG. 11, the encapsulated ibuprofen soft chewable tablets exhibited less astringency and weaker mouth burn.

No additional negative attributes were detected during the aftertaste evaluation, and bitterness did not increase relative to the control.

This demonstrates that the benefits of encapsulation persist beyond the initial chew and extend into the aftertaste of encapsulated ibuprofen soft chewable tablets.

### Example 7: Taste Masking of APIs in Alternative Gelling Systems Using Encapsulation Technology

This example demonstrated the application of encapsulation technology to improve the palatability of active pharmaceutical ingredients (APIs). Acetaminophen (APAP) and ibuprofen were encapsulated in various chewable compositions. The study evaluated the effectiveness of encapsulation in reducing bitterness and enhancing sweetness and fruity flavor across different gelling matrices, including soft chews, gelatin, and carrageenan-based systems.

Four groups of samples were prepared:

Group A: Soft chews with encapsulated APAP, free APAP, and placebo.

Group B: Gelatin-based soft chewable tablets with encapsulated and free APAP.

Group C: Carrageenan-based soft chewable tablets with encapsulated and free APAP.

Group D: Gelatin-based soft chewable tablets with encapsulated and free ibuprofen.

Table 7 provides the details of the samples prepared.

TABLE 7
Samples prepared and tested.

| Group | Sample # | Description | Dose | Flavor |
|---|---|---|---|---|
| A | 1 | Encapsulated APAP | 500 mg | Redberry |
| | | Soft Chew | | |
| A | 2 | APAP Soft Chew | 500 mg | Redberry |
| A | 3 | Placebo Soft Chew | 0 mg | Redberry |
| B | 4 | Encapsulated Gelatin | 500 mg | Redberry |
| | | APAP soft chewable | | |
| | | tablet | | |
| B | 5 | Gelatin APAP soft | 500 mg | Redberry |
| | | chewable tablet | | |
| C | 6 | Encapsulated | 500 mg | Raspberry |
| | | Carrageenan APAP | | Orange Lime |
| | | soft chewable tablet | | |
| C | 7 | Carrageenan APAP | 500 mg | Raspberry |
| | | soft chewable tablet | | Orange Lime |
| D | 8 | Encapsulated | 200 mg | Peach |
| | | ibuprofen soft | | Raspberry |
| | | chewable tablet | | |
| D | 9 | Ibuprofen soft | 200 mg | Peach |
| | | chewable tablet | | Raspberry |

Ibuprofen may cause burning sensations in the mouth and throat. It is often described as astringent but not very bitter. Each sample was flavored with redberry or raspberry-orange-lime profiles and evaluated by a trained sensory panel using the Descriptive Analysis method described in Example 1. Taste attributes were assessed at two time points: initial chew (15 seconds) and aftertaste (30 seconds post expectoration).

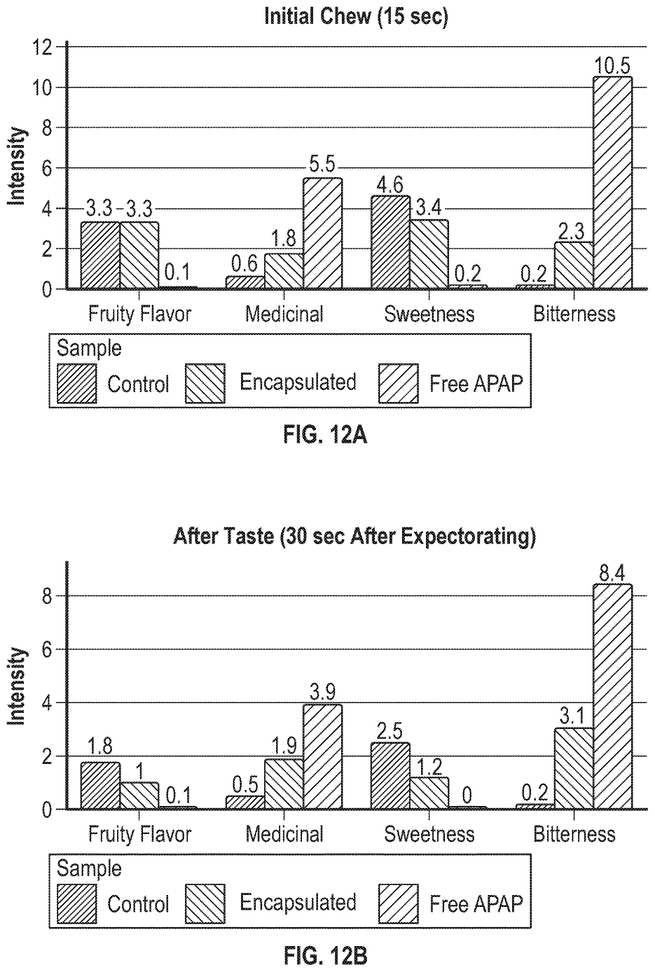

Group A: Soft Chews with APAP. FIG. 12A and FIG. 12B show initial and aftertaste sensory scores for fruity flavor, sweetness, bitterness, and medicinal notes in Group A soft chews. As shown in FIG. 12A and FIG. 12B, encapsulated APAP soft chews exhibited significantly lower bitterness and higher sweetness and fruity flavor compared to free APAP soft chew. As shown in FIG. 12A, the free APAP sample exhibited substantially elevated medicinal and bitter attributes, while the encapsulated APAP sample demonstrated reduced bitterness and medicinal intensity relative to free APAP, with fruity flavor and sweetness comparable to the control. This confirms improved palatability, with fruity flavor and sweetness similar to the control chew. However, medicinal bitterness was not completely eliminated and remained slightly higher than the placebo. As shown in FIG. 12B, the free APAP sample exhibits markedly elevated medicinal (3.94) and bitter (8.44) intensities relative to both the control and encapsulated samples 30 seconds after expectorating, while the encapsulated APAP sample demonstrates substantially reduced bitterness and medicinal aftertaste, with sweetness and fruity flavor closely aligned to the control.

Table 8 provides a statistical summary of the data.

TABLE 8

| | | Total | | | | |
|---|---|---|---|---|---|---|
| Variable | Sample | Count | Mean | StDev | Range | Grouping |
| Fruity Flavor | Control | 16 | 1.75 | 0.93 | 3 | A |
| | Encapsulated | 16 | 1 | 0.97 | 3 | B |
| | Free APAP | 16 | 0.06 | 0.25 | 1 | C |
| Medicinal | Control | 16 | 0.5 | 0.73 | 2 | A |
| | Encapsulated | 16 | 1.88 | 1.45 | 4 | B |
| | Free APAP | 16 | 3.94 | 2.24 | 8 | C |
| Sweetness | Control | 16 | 2.50 | 1.10 | 4 | A |
| | Encapsulated | 16 | 1.19 | 1.11 | 3 | B |
| | Free APAP | 16 | 0 | 0 | 0 | C |
| Bitterness | Control | 16 | 0.19 | 0.40 | 1 | A |
| | Encapsulated | 16 | 3.06 | 1.77 | 5 | B |
| | Free APAP | 16 | 8.44 | 2.90 | 10 | C |

Group B: Gelatin-Based Soft Chewable Tablets with APAP

Two-sample t-tests revealed that encapsulated APAP significantly reduced bitterness and improved sweetness and fruity flavor compared to free APAP (Tables 9 and 10). While medicinal notes were present in both samples, the difference was not statistically significant.

TABLE 9
Group B, Initial.

| | | Total | | | | |
|---|---|---|---|---|---|---|
| Variable | Sample | Count | Mean | StDev | Range | P value |
| Fruity Flavor | Encapsulated | 13 | 2.54 | 1.05 | 4 | <0.05 |
| | Free APAP | 13 | 1.69 | 0.95 | 3 | |
| Medicinal | Encapsulated | 13 | 3.08 | 1.44 | 4 | >0.05 |
| | Free APAP | 13 | 3.46 | 1.66 | 5 | |
| Sweetness | Encapsulated | 13 | 3.00 | 1.41 | 5 | <0.05 |
| | Free APAP | 13 | 1.69 | 1.11 | 3 | |
| Bitterness | Encapsulated | 13 | 4.31 | 1.03 | 3 | <0.05 |
| | Free APAP | 13 | 6.92 | 2.69 | 9 | |

TABLE 10
Group B, After taste

| | | Total | | | | |
|---|---|---|---|---|---|---|
| Variable | Sample | Count | Mean | StDev | Range | P value |
| Fruity Flavor | Encapsulated | 13 | 0.54 | 0.78 | 2 | >0.05 |
| | Free APAP | 13 | 0.08 | 0.28 | 1 | |
| Medicinal | Encapsulated | 13 | 2.69 | 1.18 | 4 | >0.05 |
| | Free APAP | 13 | 3 | 1.63 | 5 | |
| Sweetness | Encapsulated | 13 | 1.31 | 0.95 | 3 | <0.05 |
| | Free APAP | 13 | 0.15 | 0.38 | 1 | |
| Bitterness | Encapsulated | 13 | 4 | 1 | 3 | <0.05 |
| | Free APAP | 13 | 6.62 | 2.02 | 7 | |

Group C: Carrageenan-Based Soft Chewable Tablets with APAP

Encapsulation in carrageenan-based systems also resulted in significantly improved taste profiles. As shown in Tables 11 and 12, bitterness and medicinal notes were substantially reduced, while sweetness and berry flavor were enhanced.

TABLE 11
Group C, Initial.

| | | Total | | | | |
|---|---|---|---|---|---|---|
| Variable | Sample | Count | Mean | StDev | Range | P value |
| Berry | Encapsulated | 18 | 3.33 | 1.88 | 8 | <0.05 |
| | Free APAP | 18 | 1.89 | 1.68 | 6 | |
| Citrus | Encapsulated | 18 | 0.94 | 1.11 | 3 | >0.05 |
| | Free APAP | 18 | 1.56 | 1.20 | 4 | |
| Medicinal | Encapsulated | 18 | 1.00 | 1.03 | 3 | <0.05 |
| | Free APAP | 18 | 4.22 | 1.80 | 6 | |
| Sweetness | Encapsulated | 18 | 5.17 | 2.41 | 10 | <0.05 |
| | Free APAP | 18 | 3.28 | 1.64 | 6 | |
| Bitterness | Encapsulated | 18 | 1.56 | 1.15 | 4 | <0.05 |
| | Free APAP | 18 | 5.11 | 2.40 | 8 | |

TABLE 12
Group C, After taste.

| | | Total | | | | |
|---|---|---|---|---|---|---|
| Variable | Sample | Count | Mean | StDev | Range | P value |
| Berry | Encapsulated | 18 | 1.50 | 1.58 | 6 | <0.05 |
| | Free APAP | 18 | 0.39 | 0.78 | 2 | |
| Citrus | Encapsulated | 18 | 0.72 | 1.27 | 5 | >0.05 |
| | Free APAP | 18 | 0.67 | 0.91 | 2 | |
| Medicinal | Encapsulated | 18 | 1.67 | 1.37 | 4 | <0.05 |
| | Free APAP | 18 | 3.28 | 1.56 | 6 | |
| Sweetness | Encapsulated | 18 | 1.83 | 1.29 | 5 | <0.05 |
| | Free APAP | 18 | 0.89 | 1.13 | 3 | |
| Bitterness | Encapsulated | 18 | 3.33 | 2.33 | 10 | <0.05 |
| | Free APAP | 18 | 5.50 | 2.12 | 7 | |

Group D: Soft Chewable Tablets with Ibuprofen

Encapsulation of ibuprofen also resulted in significantly improved taste profiles as well as reduction in mouth burn. As shown in Table 13, bitterness, astringency, and mouth burn were substantially reduced.

TABLE 13

| | Control - | Test - | p- |
|---|---|---|---|
| | Free | Encapsulated | value |
| Initial Chew | | | |
| Fruity Flavor | 3.2 | 3.4 | 0.38 |
| Chemical Feeling Factor- | 1.1 | 1.1 | 1.00 |
| Metallic | | | |
| Chemical Feeling Factor- | 2.3 | 1.3 | 0.06 |
| Astringent (mouthdrying) (*) | | | |
| Chemical Feeling Factor- | 2.7 | 1.5 | 0.03 |
| Mouthburn (**) | | | |
| Basic Taste-Sweet | 2.1 | 2.9 | 0.20 |
| Basic Taste-Bitter (***) | 1.6 | 0.6 | 0.01 |
| Aftertaste | | | |
| Fruity Flavor | 0.8 | 1.4 | 0.14 |
| Chemical Feeling Factor- | 1.6 | 0.8 | 0.06 |
| Metallic (*) | | | |
| Chemical Feeling Factor- | 2.4 | 1.9 | 0.14 |
| Astringent (mouthdrying) | | | |
| Chemical Feeling Factor- | 2.7 | 1.4 | 0.02 |
| Mouthburn (**) | | | |
| Basic Taste-Sweet | 0.4 | 0.9 | 0.17 |
| Basic Taste-Bitter | 1.6 | 1.1 | 0.20 |

Significant at alpha 0.1 (*)
Significant at alpha 0.05 (**)
Significant at alpha 0.01 (***)

What is claimed is:

1. A method of making a chewable composition for masking the taste of a primary ingredient, the method comprising:
combining a sweetener, a hydrocolloid, and a solvent to form a homogeneous mixture;
adding an encapsulated primary ingredient to the homogeneous mixture while mixing, the primary ingredient selected from the group consisting of an active pharmaceutical ingredient, dietary supplement, food additive, and any combination thereof;
conditioning the homogeneous mixture to a temperature from about 40° C. to about 95° C.;
depositing the homogeneous mixture into molds; and
cooling the deposited homogenous mixture to form the chewable composition.

2. The method of claim 1, wherein the combining is conducted at room temperature.

3. The method of claim 1, wherein the combining further includes combining a non-encapsulated active pharmaceutical ingredients color additive, humectant, gelling agent, or pH modifier, or any combination thereof to form the homogeneous mixture.

4. The method of claim 1, wherein the conditioning comprises heating the homogeneous mixture.

5. The method of claim 1, wherein the conditioning comprises cooling the homogeneous mixture.

6. The method of claim 1, wherein homogeneous mixture is conditioned to a temperature from about 40° C. to about 70° C.

7. The method of claim 6, further comprising maintaining the temperature of the homogenous mixture after the conditioning for a period of about 40 minutes or longer.

8. The method of claim 7, wherein the temperature of the homogeneous mixture after the conditioning is maintained for a period of 1 hour.

9. The method of claim 7, wherein the temperature of the homogeneous mixture after the conditioning is maintained for a period of 2 hours.

10. The method of claim 1, wherein the encapsulated primary ingredient comprises an active pharmaceutical ingredient.

11. The method of claim 10, wherein the active pharmaceutical ingredient includes a non-steroidal anti-inflammatory drug (NSAID) including acetylsalicylic acid, ibuprofen, naproxen, ketoprofen, flurbiprofen, fenoprofen, diclofenac, indomethacin, ketorolac, etodolac, sulindac, or tolmetin.

12. The method of claim 1, further comprising adding a coloring agent or flavoring agent to the homogeneous mixture prior to depositing the homogeneous mixture into molds.

13. The method of claim 1, wherein the hydrocolloid comprises carrageenan, gelatin, pectin, agar, starch, locust bean gum, konjac, gum Arabic, sago, or any combination thereof.

14. The method of claim 1, wherein the sweetener comprises glucose, fructose, galactose, psicose, isomaltulose, tagatose, sucrose, maltose, trehalose, sorbose, raffinose, a sugar alcohol, tagatose, corn syrup, corn syrup solids, or any combination thereof.

15. The method of claim 1, further comprising sanding the chewable composition with sugar.

16. The method of claim 1, further comprising coating the chewable composition with oils, waxes, maltodextrin, starch, or any combination thereof.

17. The method of claim 1, wherein the solvent is water.

18. A method of making a chewable composition for masking the taste of a primary ingredient, the method comprising:
combining a sweetener and a solvent to form a homogeneous mixture;
adding an encapsulated primary ingredient to the homogeneous mixture while mixing, the primary ingredient selected from the group consisting of an active pharmaceutical ingredient, dietary supplement, food additive, and any combination thereof;
conditioning the homogeneous mixture to a temperature from about 40° C. to about 95° C.; and
forming the homogeneous mixture into shapes to form the chewable composition.

19. The method of claim 18, wherein homogeneous mixture is conditioned to a temperature from about 40° C. to about 70° C.

20. The method of claim 19, further comprising maintaining the temperature of the homogenous mixture after the conditioning for a period of about 40 minutes or longer.
